(19) United States
(12) Patent Application Publication
(10) Pub. No.: US 2021/0071232 A1
(43) Pub. Date: Mar. 11, 2021

(54) RNA PRESERVATION SOLUTION AND METHODS OF MANUFACTURE AND USE

(71) Applicant: Spectrum Solutions L.L.C., Draper, UT (US)

(72) Inventor: Federico Gaeta, Torre Molinos (ES)

(21) Appl. No.: 16/958,552

(22) PCT Filed: Nov. 14, 2019
(86) PCT No.: PCT/US19/61525

§ 371 Date: Jun. 26, 2020

Related U.S. Application Data
(60) Provisional application No. 62/767,296, filed on Nov. 14, 2018.

(51) Int. Cl. C12Q 1/6806

(52) U.S. Cl. CPC C12Q 1/6806

(57) ABSTRACT

Disclosed is nucleic acid preserving compositions and methods of manufacturing and using the same. Compositions include a carrier, a chaotropic agent, a buffering agent, a chelating agent, a surfactant, an alcohol, and a reducing agent. Compositions as aqueous solutions can include water as a carrier. Preferred embodiments include RNAse-free water, lithium chloride, sodium citrate, EDTA, CTAB or SLS, SDA 3C, and TCEP, with HCl optionally added to adjust pH. Some embodiments include a colored dye as a visual indicator. Methods of manufacturing include combining the components into a mixture, such as an aqueous solution. Methods of use include providing a biological sample that includes nucleic acid, preferably RNA, and contacting the biological sample with the composition. Kits include a biological sample collection apparatus and the composition. The composition is optionally disposed in a portion of the collection apparatus.

### BACKGROUND

### 1. Technical Field

The present disclosure relates to preserving nucleic acid, particularly ribonucleic acid (RNA). Specifically, the present disclosure relates to compositions and methods for preserving human RNA in a biological sample, such as saliva, for further analysis.

### 2. Related Technology

Nucleic acid can be extracted from biological samples, such as bodily fluids and tissue sample, that include cellular and/or cell-free nucleic acids. Extracted nucleic acid (e.g., RNA) can be used for a variety of analytical purposes, including gene expression profiling. Nucleic acid-containing biological samples often need to be properly processed for specific types of nucleic acid analysis. Analytical techniques such as RNA sequencing (RNA-Seq), microarray, expressed sequence tag (EST), reverse transcription polymerase chain reaction (RT-PCR), fluorescent in situ hybridization (FISH), and northern blot analyses, for example, may require specific processing or pre-processing steps that depend on the specific platform to be used.

In some cases, nucleic acid-containing biological samples may need to be processed, or steps may need to be taken, in order to stabilize the sample or nucleic acid thereof. RNA, in particular, is known to be highly unstable and/or sensitive to degradation under certain conditions (e.g., in solution and/or when exposed to nuclease, unfavorable temperatures, UV light, and/or various chemicals). Stabilizing or preserving reagents (e.g., solutions) are often added to nucleic acid-containing biological samples during storage and processing steps to ensure survival of at least a portion of the nucleic acids, particularly RNA, until analysis thereof can be performed. Without being bound to any particular theory, RNA preservation or stabilization is generally considered to be much more difficult than DNA preservation or stabilization.

Existing stabilizing solutions may not be optimal for stabilizing RNA from certain biological samples, such as saliva, and/or for certain types of analytical techniques or devices for performing the same. For instance, an RNA stabilizing solution formulated for optimal or suitable qRT-PCR analysis may not be optimal or suitable for analysis in EST platforms. In some cases, improper formulation may produce or lead to analytical artifacts, high background signal (or noise), contamination and/or retention of microbial nucleic acids, which may obscure human nucleic acid results, and/or may reduce the total potential yield, purity, and/or stability of human nucleic acid in a biological sample.

Existing stabilizing solutions may also be deficient in controlling microbial life. Biological sample, such as saliva, tissues and cells, can include and/or become contaminated with one or more microbes (e.g., bacteria, fungi, etc.). These microbes contain nucleic acids that may interfere or be detected along with the nucleic acid of the host or source of the biological sample. Preservation solutions may inadvertently stabilize microbial nucleic acids or even permit the growth of the microorganisms.

Accordingly, there continues to be a need for RNA stabilizing solutions that are suitable for a variety of analytical techniques and/or devices, provide better overall yield, purity, and/or stability of RNA in a biological sample, as compared to existing products, enhance the quality of the biological sample, such as by controlling microbial growth and/or reducing microbial nucleic acid contamination, as compared to existing products, and combinations thereof.

### BRIEF DESRIPTION OF THE DRAWINGS

Various embodiments of the present disclosure will now be discussed with reference to the appended drawings. It is appreciated that these drawings depict only typical embodiments of the presents disclosure and are therefore not to be considered limiting of its scope.

FIG. 1 illustrates average yield of RNA for various RNA preservation compositions according to an embodiment of the present disclosure.

FIG. 2 illustrates average RNA quality score (RQS) for various RNA preservation compositions according to an embodiment of the present disclosure.

FIGS. 3A, 3B, and 3C illustrate Yield, Purity, Fidelity Results, respectively, of RNA extracted from saliva samples immediately after collection in an RNA preservation composition according to an embodiment of the present disclosure.

FIGS. 4A, 4B, and 4C illustrate Yield, Purity, Fidelity Results, respectively, of RNA extracted from saliva samples after being stored at room temperature for 48 hours in an RNA preservation composition according to an embodiment of the present disclosure.

FIGS. 5A, 5B, and 5C illustrate Yield, Purity, Fidelity Results, respectively, of RNA extracted from saliva samples after being stored frozen for 48 hours in an RNA preservation composition according to an embodiment of the present disclosure.

### BRIEF SUMMARY

Embodiments of the present disclosure solve one or more of the foregoing or other problems in the art with nucleic acid (e.g., RNA) preservation, stabilization, and/or preparation compositions, kits comprising the same, and methods of manufacturing and using the same. For instance, some embodiments of the present disclosure include compositions for preserving, stabilizing, and/or preparing nucleic acid (e.g., RNA) in a biological sample. The biological sample can be saliva or another bodily fluid, in certain embodiments. The compositions can be suitable for use in a variety of analytical techniques and devices. Specifically, the compositions can be formulated to be compatible for use in a variety of analytical techniques and devices. The compositions can yield high amounts of nucleic acid (e.g., RNA) for subsequent analysis and/or processing. For example, the composition can yield high amounts of human nucleic acid (e.g., RNA), preferably and/or optionally with low amounts of microbial (e.g., bacterial) nucleic acid (e.g., RNA and/or DNA) for subsequent analysis and/or processing. The composition can comprise a solution or water-based (e.g., aqueous) liquid, optionally (light) blue in color. The composition can be suitable for use in the stabilization of human nucleic acid (e.g., RNA) and, preferably, prevention of bacterial contamination and/or growth and for (long-term) sample storage.

In at least one aspect, an embodiment of the present disclosure includes a ribonucleic acid (RNA) preservation composition. The preservation composition can comprise a carrier, a buffer or buffering agent, and a (metal) chelating agent. The composition can also include one or more additional reagents, preferably selected from the group consisting of a chaotropic agent, a detergent or a surfactant, an alcohol, and a reducing agent. The composition can also have a pH of 4-7 and/or an acid q.s. to a pH of 4-7, preferably a pH of 5.5. The composition can also include an optional visual indicator.

In at least one aspect, an embodiment of the present disclosure includes an RNA preservation composition, comprising a carrier, a buffering agent, a chelating agent, and a chaotropic agent. The composition can also include one or more additional reagents, preferably selected from the group consisting of a detergent or a surfactant, an alcohol, and a reducing agent. The composition can also have a pH of 4-7 and/or an acid q.s. to a pH of 4-7, preferably a pH of 5.5. The composition can also include an optional visual indicator.

In at least one aspect, an embodiment of the present disclosure includes an RNA preservation composition, comprising a carrier, a buffering agent, a chelating agent, a chaotropic agent, and a detergent or a surfactant. The composition can also include one or more additional reagents, preferably selected from the group consisting of an alcohol and a reducing agent. The composition can also have a pH of 4-7 and/or an acid q.s. to a pH of 4-7, preferably a pH of 5.5. The composition can also include an optional visual indicator.

In at least one aspect, an embodiment of the present disclosure includes an RNA preservation composition, comprising a carrier, a buffering agent, a chelating agent, a chaotropic agent, and an alcohol. The composition can also include one or more additional reagents, preferably selected from the group consisting of a detergent or a surfactant and a reducing agent. The composition can also have a pH of 4-7 and/or an acid q.s. to a pH of 4-7, preferably a pH of 5.5. The composition can also include an optional visual indicator.

In at least one aspect, an embodiment of the present disclosure includes an RNA preservation composition, comprising a carrier, a buffering agent, a chelating agent, a chaotropic agent, a reducing agent, and an alcohol. The composition can also include a detergent or a surfactant. The composition can also have a pH of 4-7 and/or an acid q.s. to a pH of 4-7, preferably a pH of 5.5. The composition can also include an optional visual indicator.

In at least one aspect, an embodiment of the present disclosure includes an RNA preservation composition, comprising a carrier, a chaotropic agent, a buffering agent, a (metal) chelating agent, a detergent (or surfactant), an alcohol, and a reducing agent. In some embodiments, an acid (or base) can be added to achieve a suitable final pH. The composition can have a pH of 4-7 and/or an acid q.s. to a pH of 4-7, preferably a pH of 5.5. The composition can also include an optional visual indicator.

In at least one aspect, an embodiment of the present disclosure includes an alcohol-free ribonucleic acid (RNA) preservation composition, comprising a carrier, a buffering agent, and a chelating agent, and, optionally, one or more reagents or ingredients selected from the group consisting of a chaotropic agent, a detergent or a surfactant, and a reducing agent. In some embodiments, an acid (or base) can be added to achieve a suitable final pH. The composition can have a pH of 4-7 and/or an acid q.s. to a pH of 4-7, preferably a pH of 5.5. The composition can also include an optional visual indicator. In some embodiments, the composition comprises a carrier, a buffering agent, a chelating agent, a chaotropic agent, a detergent or a surfactant, and a reducing agent. In some embodiments, an acid (or base) can be added to achieve a suitable final pH of 4-7, preferably a pH of 5.5.

In various aspects, or embodiments thereof, the composition can be or comprise a liquid, or in liquid form. Preferably, the composition can be or comprise a solution.

In some embodiments, the carrier can be a fluid or liquid carrier. In a preferred embodiment, the carrier is an aqueous carrier. The carrier can comprise water, preferably filtered, purified, distilled, and/or deionized, RNAse-free water.

In some embodiments, the composition can comprise about 0.1%-10%, w/w, preferably about 0.5-5%, w/w, more preferably about 1%-2%, w/w, of the buffering agent. In some embodiments, the buffering agent can be or comprise sodium citrate (e.g., trisodium citrate dihydrate; C₆H₅O₇Na₃.2H₂O)) or another suitable buffering agent.

In some embodiments, the composition can comprise about 1%-10%, w/w, preferably about 2-8%, w/w, more preferably about 3%-7%, w/w, still more preferably about 3.33%-6.66%, w/w, of the chaotropic agent. In some embodiments, the chaotropic agent can be or comprise lithium chloride (LiCl) or another suitable chaotropic agent.

In some embodiments, the composition can comprise about 0.01-1%, w/w, preferably about 0.05-0.5%, w/w, more preferably about 0.1%-0.3%, w/w, still more preferably about 0.2%, w/w, of the (metal) chelating agent. In some embodiments, the (metal) chelating agent can be or comprise ethylenediaminetetraacetic acid (EDTA), preferably as EDTA disodium salt, more preferably as EDTA disodium dihydrate (a.k.a. Edetate Disodium Dihydrate) or another suitable (metal) chelating agent.

In some embodiments, the composition can comprise about 1%-10%, w/w, preferably about 2-8%, w/w, more preferably about 3%-5%, w/w, still more preferably about 4%, w/w, of the detergent (or surfactant). In some embodiments, the detergent (or surfactant) can be or comprise N-lauroylsarcosine sodium salt (a.k.a. sodium lauroyl sarcosinate (SLS), Sarkosyl NL, N-Dodecanoyl-N-methylglycine sodium salt) or another suitable detergent (or surfactant).

In some embodiments, the composition can comprise about 0.5%-30%, w/w, preferably about 1-20%, w/w, more preferably about 2%-15%, w/w, still more preferably about 5-10%, w/w, of the alcohol. In some embodiments, the alcohol can be or comprise ethanol, preferably a specially denatured alcohol (SDA) or a mixture of ethanol and isopropanol, more preferably a mixture of about 95% ethanol, v/v and about 5% isopropanol, v/v, or SDA 3C, or another suitable alcohol.

In some embodiments, the composition can comprise 0.01-1%, w/w, preferably about 0.05-0.5%, w/w, more preferably about 0.1%-0.5%, w/w, still more preferably about 0.1%-0.3%, w/w, still more preferably about 0.2%, w/w, of the reducing agent. In some embodiments, the reducing agent can be or comprise tris(2-carboxyethyl)phosphine hydrochloride (TCEP), or another suitable reducing agent.

In some embodiments, the acid, if any, can be or comprise hydrochloric acid. In some embodiments, the acid can be included q.s. to a pH of 4-7, preferably 4.5-6.5, more preferably 5-6, still more preferably 5.2-5.8, still more preferably 5.4-5.6, most preferably 5.5.

In some embodiments, the optional visual indicator can be or comprise a coloring agent, such as a dye (e.g., FD&C Blue No. 1).

In some embodiments, the composition can be (suitable) for use in preserving human RNA, preferably from a human biological sample. The human biological sample can be a fluid sample, preferably human saliva or a human saliva sample. Thus, in certain aspects, embodiments of the present disclosure can comprise human RNA preservation composition or RNA preservation composition for use in preserving human RNA.

In some embodiments, the composition can comprise about 1%-10%, w/w, of the chaotropic agent, about 0.1-10%, w/w, of the buffering agent, about 0.01-1%, w/w, of the chelating agent, about 1%-20%, w/w, of the surfactant, about 1%-20%, w/w, of the alcohol, and/or about 0.01-1%, w/w, of the reducing agent, and a pH of 4-7, preferably about 5.5.

In some embodiments, the composition can comprise about 3%-7%, w/w, of the chaotropic agent, about 0.5-5%, w/w, of the buffering agent, about 0.05-0.5%, w/w, of the chelating agent, about 2%-6%, w/w, of the surfactant, about 2%-15%, w/w, of the alcohol, and/or about 0.1-0.5%, w/w, of the reducing agent, and a pH of 4-7, preferably about 5.5.

In some embodiments, the composition can comprise about 3.33%-6.66%, w/w, of the chaotropic agent, about 1-2%, w/w, of the buffering agent, about 0.2%, w/w, of the chelating agent, about 4%, w/w, of the surfactant, about 5%-10%, w/w, of the alcohol, and/or about 0.2%, w/w, of the reducing agent, and a pH of 4-7, preferably about 5.5.

In some embodiments, the composition can comprise about 2-8%, w/w, of the buffering agent, about 1%-10%, w/w, of the chaotropic agent, about 0.01-1%, w/w, of the chelating agent, about 1%-5%, w/w, of the surfactant, about 5%-30%, w/w, of the alcohol, and/or about 0.01-1%, w/w, of the reducing agent, and a pH of 4-7.

In some embodiments, the composition can comprise about 3%-7%, w/w, of the chaotropic agent, about 4-6%, w/w, of the buffering agent, about 0.1-0.3%, w/w, of the chelating agent, about 2%-4%, w/w, of the surfactant, about 9%-21%, w/w, of the alcohol, and/or about 0.1-0.25%, w/w, of the reducing agent, and a pH of 4-7.

In some embodiments, the composition can comprise about 3.33%-6.65%, w/w, of the chaotropic agent, about 5.99%, w/w, of the buffering agent, about 0.2%, w/w, of the chelating agent, about 2.99%, w/w, of the surfactant, about 9.98%-19.96%, w/w, of the alcohol, and/or about 0.17%, w/w, of the reducing agent, and a pH of 4-7.

Various embodiments can include the carrier q.s. to 100%, w/w.

In at least one embodiment, the composition can comprise, or consist essentially of, about 3.33%-6.66%, w/w, lithium chloride (LiCl), about 1-2%, w/w, trisodium citrate dihydrate, about 0.2%, w/w, ethylenediaminetetraacetic acid (EDTA) disodium dihydrate, about 4%, w/w, N-lauroylsarcosine sodium salt (SLS) or cetyltrimethylammonium bromide (CTAB), preferably CTAB, about 5%-10%, w/w, alcohol, preferably consisting essentially of about 95%, v/v, ethanol and about 5%, v/v, isopropanol, about 0.2%, w/w, tris(2-carboxyethyl)phosphine hydrochloride (TCEP), optionally, a colored dye, and RNAse-free water q.s. to 100%, with a a pH of 4-7, preferably about 5.5.

Some embodiments can further include about 0.00037%, w/w, visual indicator (e.g., FD&C Blue No. 1) or equivalent thereof (e.g., 0.185%, w/w, of a 0.2%, w/w, visual indicator concentrate (e.g., in water)).

In some embodiments, the amount of one or more (e.g., each) of the components can be±10% (where 20% w/w of a component or ingredient at “+/−10%” implies from 18% w/w to 22% w/w, not a range of 10% w/w to 30% w/w), preferably±9%, more preferably ±8%, still more preferably±7%, still more preferably±6%, still more preferably±5%, still more preferably±4%, still more preferably±3%, still more preferably±2%, still more preferably±1%.

In some embodiments, the composition can have a pH of 4-7, preferably a pH of 4.5-7, 4.5-6.5, 5-7, or 5-6.5, still more preferably a pH of 5-6, still more preferably a pH of 5.2-5.8, still more preferably a pH of 5.4-5.6, most preferably a pH of 5.5.

One or more embodiments can be (substantially) free or devoid of (additional or any) antimicrobial(s) (e.g., bactericidal and/or bacteriostatic) agent(s) (e.g., besides or other than the alcohol(s), chaotropic agent(s), surfactant(s)/detergent(s), and/or reducing agent(s)). One or more embodiments can be (substantially) free or devoid of (additional or any) ribonuclease inhibitor(s), or inhibitor(s) of ribonuclease (e.g., besides or other than the chaotropic agent(s)). One or more embodiments can be (substantially) devoid of (any) a protease(s), proteinase K, and/or protease inhibitor(s).

One or more embodiments can be (substantially) free or devoid of dithiothreitol (DTT) or β-mercaptoethanol (BME). One or more embodiments can be (substantially) free or devoid of imidazolium salt(s). One or more embodiments can be (substantially) free or devoid of DNAse. One or more embodiments can be (substantially) free or devoid of guanidine thiocyanate, guanidine isocyanate, and guanidine hydrochloride. One or more embodiments can be (substantially) free or devoid of SDS and/or SLS. One or more embodiments can be (substantially) free or devoid of Tris, Tris-HCl, Trizma® base, citrate, IVIES, BES, Bis-Tris, HEPES, MOPS, Bicine, Tricine, ADA, ACES, PIPES, bicarbonate, phosphate, TAE, TBE, sodium borate, and/or sodium cacodylate (buffer). One or more embodiments can be (substantially) free or devoid of methanol, n-propanol, isopropanol, n-butanol, trifluoroethanol, phenol, or 2,6-di-tert-butyl-4-methylphenol.

Some embodiments include a method of stabilizing nucleic acid (e.g., RNA). The method can comprise contacting a biological sample containing RNA with a composition of the present disclosure (as described herein). The biological sample can comprise human saliva.

In some embodiments, the method can include providing a biological sample containing the nucleic acid (e.g., RNA) and combining a composition of the present disclosure with the biological sample. The method can also include other processing steps known in the art. An embodiment of the present disclosure includes a method of stabilizing nucleic acid (e.g., human nucleic acid, such as human RNA). An embodiment comprises contacting a biological sample containing the nucleic acid (e.g. RNA) with a composition of the present disclosure. In an embodiment, the biological sample comprises bodily fluid, such as (human) saliva, blood, urine, etc.. In another embodiment, the biological sample comprises biological tissue or cells.

Some embodiments include a biological sample preservation kit. The kit can comprise a sample collection apparatus and a composition of the present disclosure, preferably disposed in a solution compartment of the sample collection apparatus.

In some embodiments, the kit can comprise a sample collection apparatus and a nucleic acid (e.g., RNA) preservation composition. The sample collection apparatus can comprise a solution compartment. The nucleic acid or RNA preservation composition can be disposed in the solution compartment. An embodiment of the present disclosure includes a kit comprising a composition of the present disclosure disposed in a portion of a sample collection apparatus.

Some embodiments include a method of manufacturing a composition of the present disclosure. The method can include combining components of the present disclosure. The method can also include other manufacturing steps known in the art. An embodiment of the present disclosure includes a method of manufacturing a nucleic acid or RNA stabilization composition. An embodiment comprises obtaining a carrier and adding to the carrier components or ingredients of a composition of the present disclosure.

Additional features and advantages of exemplary embodiments of the present disclosure will be set forth in the description which follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### DETAILED DESCRIPTION

Before describing various embodiments of the present disclosure in detail, it is to be understood that this disclosure is not limited to the specific parameters and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments, and is not necessarily intended to limit the scope of the present disclosure and/or the claimed invention.

While the detailed description is separated into sections, the section headers and contents within each section are not intended to be self-contained descriptions and embodiments. Rather, the contents of each section within the detailed description are intended to be read and understood as a collective whole where elements of one section may pertain to and/or inform other sections. Accordingly, embodiments specifically disclosed within one section may also relate to and/or serve as additional and/or alternative embodiments in another section having the same and/or similar systems, devices, methods, and/or terminology.

### Abbreviated List of Defined Terms

To assist in understanding the scope and content of the foregoing and forthcoming written description and appended claims, a select few terms are defined directly below. Unless defined otherwise, all technical and scientific terms used herein have the same meaning as commonly understood by one of ordinary skill in the art to which the present disclosure pertains.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U. S.P.Q. 461, 463 (CCPA 1976) (emphasis in the original); see also MPEP § 2111.03. Thus, the term “consisting essentially of” when used in a claim of this disclosure is not intended to be interpreted to be equivalent to “comprising.”

The term “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU, dUTP, 7-deaza-dGTP), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “sample,” “biological sample,” and the like refers to an animal; a tissue or organ from an animal; a cell (either within a subject, taken directly from a subject, or a cell maintained in culture or from a cultured cell line); a cell lysate (or lysate fraction) or cell extract; a solution containing one or more molecules derived from a cell, cellular material, or viral material (e.g. a polypeptide or nucleic acid); or a solution containing a naturally or non-naturally occurring nucleic acid, which is or can be assayed as described herein. A sample may also be any bodily fluid or excretion that contains one or more cells, cell components, or nucleic acids, including, but not limited to cellular, nuclear, or cell-free nucleic acids.

By “bodily fluid” is meant a naturally occurring fluid, including without limitation a liquid, semi-solid, aerated liquid, liquid-gas mixture, and so forth, from an animal (e.g., human or non-human animal). Such bodily fluids can include, but are not limited to, saliva, sputum, serum, plasma, blood, urine, mucus, perspiration, tears or other ophthalmic fluids, otic fluids, puss (e.g., from a blister or sore), gastric fluids or juices, fecal fluids, pancreatic fluids or juices, semen, products of lactation or mensuration, spinal fluid, fluid bone marrow, or lymph.

By “sputum” is meant that mucoid matter contained in or discharged from the nasal or buccal cavity of a mammal. Sputum, as used herein, generally includes saliva and discharges from the respiratory passages, including the lungs.

By “saliva” is meant the secretion, or combination of secretions, from any of the salivary glands, including the parotid, submaxillary, and sublingual glands, optionally mixed with the secretion from the buccal glands.

By “mucoid” is meant any bodily fluid containing mucin.

By “mucin” is meant any mucoprotein that raises the viscosity of the medium surrounding the cells that secrete it.

As used herein, the term “about,” with regard to a value, means +/−10% of the stated value or amount represented thereby. For instance, throughout the present disclosure, the term “about” is used in connection with a percent concentration or composition of a component or ingredient (e.g., in a mixture, such as a fluid or liquid mixture, aqueous mixture, solution, etc., optionally or preferably measured as a w/w percent, w/v percent, v/v percent, etc.). In such instance, the term “about” and/or the term “+/−10%” implies and/or includes +/−10% of the stated numeric value, as opposed to +/−10 percentage points of the recited percent. By way of example, where 20% w/w of a component or ingredient reflects 20 g of the component or ingredient per 100 mL of total mixture, the term “about” and/or the term “+/−10%” implies and/or includes a recited range from 18 g to 22 g (i.e., from 18% w/w to 22% w/w), not a range of 10% w/w to 30% w/w. Alternatives for so-called “about” values and/or +/−10% include +/−1%, +/−2%, +/−3%, +/−4%, +/−5%, +/−6%, +/−7%, +/−8%, or +/−9% of the stated value, each of which is contemplated as a suitable alternative to or substitute for the term “about” or the use of +/−10% herein.

As used herein, the terms “approximately” and “substantially” represent or imply an (or any) amount close to the stated amount (e.g., that still performs a desired function or achieves a (desired or expected) result). For example, the terms “approximately” and “substantially” may refer to an amount that is within, or less than, 10%, 5%, 1%, 0.1%, 0.01%, or other percent of a stated amount. As used herein, the term “substantially devoid” means (1) an undetectable or unquantifiable amount, (2) less than or below an amount generally considered by those skilled in the art to reflect a detectable or quantifiable amount, and/or (3) less than or below an amount generally considered by those skilled in the art to be functional or able to achieve a (desired or expected) result (e.g., less than 10%, 5%, 1%, 0.1%, 0.01%, or other percent).

By “Quantum satis” (also referred to as “q.s.” or “qs”) is meant the amount that is enough. Accordingly, a component or ingredient “qs 100%,” “provided at qs 100%,” or “qs to 100%” indicates that the component or ingredient is provided or included in an amount sufficient to complete the composition or to bring the total (of all components, whether recited or not) to 100%. It is noted, however, that a (final) component or ingredient “qs 100%,” “provided at qs 100%,” or “qs to 100%” does not indicate that the mixture consists of, consists essentially of, or only contains the components listed or recited immediately before the “qs 100%” component. In other words, “qs 100%,” and similar terms, is meant to be an open-ended expression indicating the source of the remainder, whatever that remainder may be.

By “alcohol” is meant a water-miscible organic compound containing a hydroxyl group, including water-miscible mixtures of hydroxyl-containing organic compounds.

By “aqueous” is meant a medium or matter that contains 30% or more water (by volume or by weight).

By “aqueous solution” is meant a solution or suspension that contains 30% or more water by volume.

By “denaturing agent” is meant a substance that alters the natural state of that to which it is added.

By “chaotropic agent” is meant a molecule that exerts chaotropic activity. As understood by those skilled in the art, molecules that exert chaotropic activity may disrupt the hydrogen-bonding network between water molecules, thereby affecting the stability of the native state of other molecules (in the solution), mainly macromolecules (proteins, nucleic acids) by weakening the hydrophobic effect. Accordingly, molecules that exert chaotropic activity may have protein-denaturing activity (or be protein denaturants).

By “antimicrobial agent” is meant a substance or group of substances which reduces the rate of growth of an organism compared to the rate of growth of the organism in their absence. A reduction in the rate of growth of an organism may be by at least 5%, more desirably, by at least 10%, even more desirably, by at least 20%, 50%, or 75%, and most desirably, by 90% or more. The definition also extends to substances which affect the viability, virulence, or pathogenicity of an organism. An antimicrobial agent can be natural (e.g., derived from bacteria or other source), synthetic, or recombinant. An antimicrobial agent can be bacteriostatic, bactericidal or both. An antimicrobial agent is bacteriostatic if it inhibits cell division without affecting the viability of the inhibited cell. An antimicrobial agent is bactericidal if it causes cell death. Cell death is commonly detected by the absence of cell growth in liquid growth medium (e.g., absence of turbidity) or on a solid surface (e.g., absence of colony formation on agar). Those of skill in the art know that a substance or group of substances which is bacteriostatic at a given concentration may be bactericidal at a higher concentration. Certain bacteriostatic substances are not bactericidal at any concentration.

As used herein, the term “composition” includes products, formulations, and mixtures, as well as devices, apparatus, assemblies, kits, and so forth. Similarly, the term “method” includes processes, procedures, steps, and so forth.

Various aspects of the present disclosure, including systems, methods, and/or products may be illustrated with reference to one or more embodiments or implementations, which are exemplary in nature. As used herein, the terms “embodiment” and “implementation” mean “serving as an example, instance, or illustration,” and should not necessarily be construed as preferred or advantageous over other aspects disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the description thereof.

As used herein, a “feature” of the present disclosure or embodiment disclosed herein refers to a property, component, ingredient, element, part, portion, (method) step, or other aspect of the subject matter at hand.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The word “or” as used herein means any one member of a particular list and also includes any combination of members of that list.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as two or more proteins. Similarly, use of a plural referent does not necessarily require a plurality of such referents, but contemplates, includes, and specifically discloses one, as well as two or more of such referents, unless the context clearly dictates otherwise.

It is noted that embodiments of the present disclosure can comprise one or more combinations of two or more of the features described herein. As used herein, “feature(s)” and similar terms can include, for example, compositions, ingredients, components, elements, members, parts, portions, systems, methods, configurations, parameters, properties, and so forth. Embodiments can include any of the features, options, and/or possibilities set out elsewhere in the present disclosure, including in other aspects or embodiments of the present disclosure. It is also noted that each of the foregoing, following, and/or other features described herein represents a distinct embodiment of the present disclosure. Features can also be combined and/or combinable with another one or more other features in any suitable combination and/or order, with or without one or more additional features included therewith or performed therebetween, to form unique embodiments, each of which is contemplated in the present disclosure. Such combinations of any two or more of such features represent distinct embodiments of the present disclosure. Accordingly, the present disclosure is not limited to the specific combinations of exemplary embodiments described in detail herein and disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. Likewise, any steps recited in any method described herein and/or recited in the claims can be executed in any suitable order and are not necessarily limited to the order described and/or recited, unless otherwise stated (explicitly or implicitly). Such steps can, however, also be required to be performed in a particular order in certain embodiments of the present disclosure.

It will also be appreciated that where two or more values, or a range of values (e.g., less than, greater than, at least, and/or up to a certain value, and/or between two recited values) is disclosed or recited, any specific value or range of values falling within the disclosed values or range of values is likewise specifically disclosed and contemplated herein. Thus, disclosure of an illustrative measurement (e.g., length, width, thickness, etc.) that is less than or equal to about 10 units or between 0 and 10 units includes, illustratively, a specific disclosure of: (i) a measurement of 9 units, 5 units, 1 units, or any other value between 0 and 10 units, including 0 units and/or 10 units; and/or (ii) a measurement between 9 units and 1 units, between 8 units and 2 units, between 6 units and 4 units, and/or any other range of values between 0 and 10 units.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

### Illustrative Embodiments

The following description of embodiments includes disclosure that is relevant to one or more embodiments of the present disclosure. Accordingly, some embodiments can include features disclosed in the following examples without departing from the scope of the present disclosure. In other words, features disclosed in the following examples can be included and/or incorporated into any one or more of the embodiments disclosed herein.

### Compositions

Some embodiments of the present disclosure include a composition. The compositions can be or comprise a ribonucleic acid (RNA) preservation composition, preferably in liquid form. The compositions can stabilize RNA in a biological sample, preferably a (human) fluid biological sample (e.g., sputum or saliva, tissue, cells, etc.) and/or render the biological sample a viable source of RNA for purification and analysis. The compositions provide the advantageous properties of chemical stabilization of nucleic acids, particularly ribonucleic acid (RNA), and the inhibition of nucleases, including ribonucleases, and microbial growth. Chemical stabilization of the nucleic acids (e.g., RNA) in a sample can be achieved through the use of buffers, acids, chelating agents, reducing agents, chaotropic agents, surfactants, and alcohol. In particular, the invention compositions of the present disclosure can advantageously lead to cleaner human RNA analysis results with respect to any contaminating microbial nucleic acid. The compositions can also provide the advantageous property of rendering the preserved sample suitable for use with a variety of analytical methods and/or devices.

Moreover, compositions of the present disclosure, when mixed with a biological sample (e.g., mucin-containing bodily fluid, tissue sample, cells, etc.) can preserve the nucleic acids (e.g., RNA) at room temperature (e.g., under ambient conditions) or below room temperature (e.g., refrigerated or frozen) for extended periods of time. Samples can also be refrigerated, but freezing of the samples before nucleic acid (e.g., RNA) recovery and purification may not be required, in some embodiments. The properties of certain composition of the present disclosure are that it (a) chemically stabilizes nucleic acids, particularly RNA, in a biological sample, (b) inhibits nucleases that may be present in the sample, and (c) is compatible with reagents used to purify and/or process (e.g., amplify, sequence, reverse transcribe, etc.) oligo- or polynucleotides.

Thus, compositions of the present disclosure can provide a “universal” preservation solution for human RNA from a fluid biological sample that may include contaminating microbes or microbial nucleic acid. In particular, compositions of the present disclosure are specifically formulated to be “universal” with respect to (1) human subject—since some subjects have more or different microbes in their saliva, for example, than others, (2) storage conditions—since some samples may be collected, transported, and/or stored at room temperature, while others are refrigerated or frozen, and/or (3) sample processing—since some RNA samples may be used for RNA sequencing, while others are processed using RT-PCR or other technologies.

### Carriers

In at least one embodiment, the composition can include a carrier. Preferably, the carrier can be a liquid carrier or solvent, more preferably an aqueous carrier or solvent, still more preferably water. Most preferably, the carrier can be or comprise purified, filtered (e.g., 0.2 micron filtered), distilled, and/or deionized, RNAse-free water. Accordingly, the composition can include a carrier. The carrier can be or comprise water, such as filtered water, purified water, distilled water, or deionized water.

In some embodiments, the composition can include a carrier qs to 100%. In some embodiments, the composition can include 10-60%, w/w, preferably 15-55%, w/w, more preferably 20-50%, w/w, still more preferably 25-45%, w/w, still more preferably 28-40%, w/w, still more preferably 30-35%, w/w, still more preferably 31-34%, w/w, still more preferably 32-33%, w/w, or any value or range of values therebetween, of the carrier.

### Chaotropic Agents

The composition can include a chaotropic agent (e.g., one or more chaotropic agents). In some embodiments, the chaotropic agent can be or comprise lithium chloride (LiCl). In some embodiments, the composition can include a chaotropic agent, consisting essentially of lithium chloride (LiCl); MW 42.39.

In some embodiments, the chaotropic agent can be or comprise guanidine (or guanidinium) or a suitable salt thereof, such as guanidine thiocyanate, guanidine chloride, guanidine hydrochloride, guanidinium iodide, guanidine isothiocyanate, guanidine hydrochloride, potassium thiocyanate, sodium iodide, sodium perchlorate, urea, and so forth. In at least one embodiment, the chaotropic agent can be or comprise thiocyanate or isothiocyanate.

In some embodiments, however, the composition can be substantially free or devoid of chaotropic agent(s) besides or other than LiCl. In some embodiments, the composition can include LiCl and be (substantially) free or devoid of chaotropic agent(s) besides or other than LiCl. In some embodiments, the composition can be substantially free or devoid of guanidine (or guanidinium) or a suitable salt thereof, such as guanidine thiocyanate, guanidine chloride, guanidine hydrochloride, guanidinium iodide, guanidine isothiocyanate, and so forth. In at least one embodiment, the composition can be substantially free or devoid of thiocyanate or isothiocyanate.

In some embodiments, the chaotropic agent can be in, have, comprise, or be provided in a dry, solid, powdered, anhydrous, and/or granular form. In some embodiments, the chaotropic agent can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the chaotropic agent can comprise or be (provided) in the form of a stock solution (e.g., in water) having any suitable concentration.

In some embodiments, the composition can include about 1-20%, w/w, preferably about 1-10%, w/w, more preferably about 3-7%, w/w, still more preferably about 3.33-6.66%, w/w, or any value or range of values therebetween, of the chaotropic agent (e.g., lithium chloride). In a preferred embodiment, the composition can include (about) 3.33%, w/w, or (about) 6.66%, w/w, of the chaotropic agent. Most preferably, the composition can include (about) 3.33%, w/w, or (about) 6.66%, w/w, lithium chloride.

In one or more embodiments, the chaotropic agent(s) can be a protein denaturant.

### Buffering Agents

The composition can include a buffering agent (or buffer, pH buffer, etc.) (e.g., one or more buffering agents (or buffers, pH buffers, etc.). In some embodiments, the buffering agent can be or comprise sodium citrate (e.g., trisodium citrate dihydrate (C₆H₅O₇Na₃. 2H₂O)). In some embodiments, the composition can include a buffering agent, consisting essentially of sodium citrate (e.g., trisodium citrate dihydrate (C₆H₅O₇Na₃. 2H₂O); MW 294.1).

In alternative embodiments, the buffering agent can be or comprise tris(hydroxymethyl)aminomethane (also known as Tris; Tris base, 2-Amino-2-(hydroxymethyl)-1,3-propanediol, THAM, Trometamol) or a suitable formulation thereof (e.g., tris(hydroxymethyl)aminomethane hydrochloride, or Tris-HCl,), Trizma® base (e.g., Tris 40% (w/w) stock solution in water), citrate, 2-(N-morpholino)ethanesulfonic acid (MES), N,N-Bis(2-hydroxyethyl)-2-aminoethanesulfonic Acid (BES), 1,3-bis(tris(hydroxymethyl)methylamino) propane (Bis-Tris), 4-(2-hydroxyethyl)-1-piperazineethanesulfonic acid (HEPES), 3-(N-morpholino)propanesulfonic acid (MOPS), N,N-bis(2-hydroxyethyl)glycine (Bicine), N-tris(hydroxymethyl)methylglycine (Tricine), N-2-acetamido-2-iminodiacetic acid (ADA), N-(2-Acetamido)-2-aminoethanesulfonic acid (ACES), piperazine-1,4-bis(2-ethanesulfonic acid) (PIPES), bicarbonate, phosphate, TAE, TBE, sodium borate, sodium cacodylate, or any combination thereof.

In some embodiments, however, the composition can be substantially free or devoid of buffering agent(s) besides or other than sodium citrate (e.g., trisodium citrate dihydrate (C₆H₅O₇Na₃.2H₂O)). In some embodiments, the composition can comprise sodium citrate (e.g., trisodium citrate dihydrate (C₆H₅O₇Na₃.2H₂O)) and be substantially free or devoid of buffering agent(s) besides or other than sodium citrate (e.g., trisodium citrate dihydrate (C₆H₅O₇Na₃.2H₂O)). In some embodiments, the composition can be substantially free or devoid of tris(hydroxymethyl)aminomethane (also known as Tris; Tris base, 2-Amino-2-(hydroxymethyl)-1,3-propanediol, THAM, Trometamol) or a suitable formulation thereof (e.g., tris(hydroxymethyl)aminomethane hydrochloride, or Tris-HCl,), Trizma® base (e.g., Tris 40% (w/w) stock solution in water), citrate, 2-(N-morpholino)ethanesulfonic acid (MES), N,N-Bis(2-hydroxyethyl)-2-aminoethanesulfonic Acid (BES), 1,3-bis(tris(hydroxymethyl)methylamino) propane (Bis-Tris), 4-(2-hydroxyethyl)-1-piperazineethanesulfonic acid (HEPES), 3-(N-morpholino)propanesulfonic acid (MOPS), N,N-bis(2-hydroxyethyl)glycine (Bicine), N-tris(hydroxymethyl)methylglycine (Tricine), N-2-acetamido-2-iminodiacetic acid (ADA), N-(2-Acetamido)-2-aminoethanesulfonic acid (ACES), piperazine-1,4-bis(2-ethanesulfonic acid) (PIPES), bicarbonate, phosphate, TAE, TBE, sodium borate, sodium cacodylate, or any combination thereof.

In some embodiments, the buffering agent can be in, have, comprise, or be provided in a dry, solid, powdered, anhydrous, and/or granular form. In some embodiments, the buffering agent can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the buffering agent can comprise or be (provided) in the form of a stock solution (e.g., in water) having any suitable concentration.

In some embodiments, the composition can include about 0.05-12%, w/w, preferably about 0.1-10%, w/w, more preferably about 0.1-8%, w/w, still more preferably about 0.1-6%, w/w, or any value or range of values therebetween, of the buffering agent, preferably sodium citrate (e.g., trisodium citrate dihydrate (C₆H₅O₇Na₃.2H₂O)). The buffering agent, preferably sodium citrate (e.g., trisodium citrate dihydrate (C₆H₅O₇Na₃. 2H₂O)), can be included in the composition at about 6% (or about 5.99%), w/w, in some embodiments, at about 1.67%, w/w, in other embodiments, and at about 0.1%, w/w, in still other embodiments.

### Chelating Agents

In at least one embodiment, the composition can include a (metal) chelating agent (or chelator) (e.g., one or more chelating agent (or chelator)). In some embodiments, the chelating agent comprises, includes, or is provide with a counter ion (e.g., sodium). In at least one embodiment, the chelating agent comprises, includes, or is provide as a hydrate (e.g., dihydrate). In some embodiments, the composition can include one or more chelating agents. The chelating agent of the composition can be selected from the group consisting of: ethylenediamine tetraacetic acid (EDTA), cyclohexane diaminetetraacetate (CDTA), diethylenetriamine pentaacetic acid (DTPA), tetraazacyclododecanetetraacetic acid (DOTA), tetraazacyclotetradecanetetraacetic acid (TETA), desferrioximine, nitrilotriacetic acid (NTA), an ethylenediamine (or 1,2-diaminoethane), or respective chelator analogs, salts, and/or hydrates thereof. Preferably, the chelating agent can be or comprise EDTA or suitable salt and/or hydrate thereof (e.g., as EDTA disodium salt, preferably as EDTA disodium (salt) dihydrate). In some embodiments, the composition can include a (metal) chelating agent, consisting essentially of EDTA or suitable salt and/or hydrate thereof (e.g., as EDTA disodium salt, preferably as EDTA disodium (salt) dihydrate; MW 372.2).

In some embodiments, the composition can include a chelating agent (or chelator), consisting essentially of EDTA or suitable salt and/or hydrate thereof (e.g., as EDTA disodium salt, preferably as EDTA disodium (salt) dihydrate).

In at least one alternative embodiment, the chelating agent can be or comprise ethylene glycol tetraacetic acid ethylene, or glycol-bis(β-aminoethyl ether)-N,N,N′,N′-tetraacetic acid (EGTA), cyclohexane diaminetetraacetate (CDTA), diethylenetriamine pentaacetic acid (DTPA), hydroxyethylethylenediaminetriacetic acid (HEDTA), tetraazacyclododecanetetraacetic acid (DOTA), tetraazacyclotetradecanetetraacetic acid (TETA), desferrioximine, nitrilotriacetic acid, or N,N-bis(carboxymethyl)glycine (NTA), an ethylenediamine (or 1,2-diaminoethane), or respective chelator analogs, salts, and/or hydrates thereof, or any combination thereof.

In some embodiments, however, the composition can be substantially free or devoid of buffering agent(s) besides or other than EDTA or suitable salt and/or hydrate thereof (e.g., EDTA disodium salt or EDTA disodium dihydrate). In some embodiments, the composition can comprise EDTA or suitable salt and/or hydrate thereof (e.g., EDTA disodium salt or EDTA disodium dihydrate) and be substantially free or devoid of buffering agent(s) besides or other than EDTA or suitable salt and/or hydrate thereof (e.g., EDTA disodium salt or EDTA disodium dihydrate). In some embodiments, the composition can be substantially free or devoid of ethylene glycol tetraacetic acid ethylene, or glycol-bis(β-aminoethyl ether)-N,N,N′,N′-tetraacetic acid (EGTA), cyclohexane diaminetetraacetate (CDTA), diethylenetriamine pentaacetic acid (DTPA), hydroxyethylethylenediaminetriacetic acid (HEDTA), tetraazacyclododecanetetraacetic acid (DOTA), tetraazacyclotetradecanetetraacetic acid (TETA), desferrioximine, nitrilotriacetic acid, or N,N-bis(carboxymethyl)glycine (NTA), an ethylenediamine (or 1,2-diaminoethane), or respective chelator analogs, salts, and/or hydrates thereof, or any combination thereof.

In some embodiments, the chelating agent (e.g., EDTA) can be in, have, comprise, or be provided in a dry, solid, powdered, anhydrous, and/or granular form. In some embodiments, the chelating agent can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the chelating agent can comprise or be (provided) in the form of a stock solution (e.g., in water) having any suitable concentration.

In some embodiments, the chelating agent can be included in the composition in a range of about 0.05%, w/w, to about 5%, w/w, preferably about 0.1% to about 4%, w/w, more preferably about 0.15% to about 3%, still more preferably about 0.2% to about 2.66%, or any value or range of values therebetween, or a molar equivalent of any of the foregoing.

In some embodiments, the chelating agent can be included in the composition in a range of about 0.05%, w/w, to about 0.5%, w/w, or about 1%, w/w, to about 5%, w/w, preferably about 0.1%, w/w, to about 0.4%, w/w, or about 1.5%, w/w, to about 4%, w/w, more preferably about 0.15% to about 0.3%, w/w, or about 2%, w/w, to about 3%, w/w, more preferably about 0.2%, w/w, or about 2.66%, w/w, or any value or range of values therebetween, or a molar equivalent of any of the foregoing. Most preferably, the composition can include (about) 0.2% EDTA or EDTA disodium (salt) dihydrate, w/w, or (about) 2.66% EDTA or EDTA disodium (salt) dihydrate, w/w, or a molar equivalent of either of the foregoing. Illustratively, the chelating agent (e.g., EDTA) can be included in the composition at about 5.4 mM, in some embodiments.

Without being bound to any theory, chelating agents can complex transition metal ions that are essential for catalyzing RNA (and DNA) degradation by nucleases. Chelating agents can also have antibacterial activity.

### Surfactants

In at least one embodiment, the composition can include a surfactant or detergent (e.g., one or more surfactant or detergent). Preferably, the surfactant can be or comprise a lauroyl sarcosinate, more preferably, N-lauroylsarcosine sodium salt or sodium lauroyl sarcosinate (SLS; Sarkosyl detergent; MW 293.4) or cetyltrimethylammonium bromide (CTAB; MW 364.4). In some embodiments, the surfactant or detergent can be or comprise a lauroyl sarcosinate, more preferably, N-lauroylsarcosine sodium salt or sodium lauroyl sarcosinate (SLS; Sarkosyl detergent). In some embodiments, the surfactant or detergent can be or comprise cetyltrimethylammonium bromide (CTAB). In some embodiments, the composition can include a surfactant or detergent, consisting essentially of lauroyl sarcosinate, more preferably, N-lauroylsarcosine sodium salt or sodium lauroyl sarcosinate (SLS; Sarkosyl detergent) and/or cetyltrimethylammonium bromide (CTAB). In some embodiments, the composition can include a surfactant or detergent, consisting essentially of lauroyl sarcosinate, more preferably, N-lauroylsarcosine sodium salt or sodium lauroyl sarcosinate (SLS; Sarkosyl detergent). In some embodiments, the composition can include a surfactant or detergent, consisting essentially of cetyltrimethylammonium bromide (CTAB).

In some embodiments, SLS can be preferable over SDS or other (less soluble, but more popular) surfactants. Without being bound to any theory, we found that SLS can be substantially more soluble than other, more popular detergent(s) (e.g., sodium dodecyl sulfate (SDS), urea, Tween, etc.), when combined with other ingredients/components in compositions of the present disclosure and/or at pHs thereof. In some embodiments, CTAB can be preferable over SDS or other (less soluble, but more popular) surfactants. Without being bound to any theory, we found that CTAB can be substantially more soluble than other, more popular detergent(s) (e.g., sodium dodecyl sulfate (SDS), urea, Tween, etc.), when combined with other ingredients/components in compositions of the present disclosure and/or at pHs thereof. In some embodiments, CTAB can be preferable over SLS.

In some embodiments, SLS can be preferable over CTAB.

In at least one alternative embodiment, the surfactant can be or comprise one or more components selected from the group consisting of urea, perchlorate, and (sodium) dodecyl sulfate (SDS). In at least one alternative embodiment, the surfactant can be or comprise one or more components selected from the group consisting of sodium dodecyl sulfate (SDS), urea, perchlorate, polysorbates (Tween™), lauryl dimethyl amine oxide, polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, Bile salts (sodium deoxycholate, sodium cholate), polyoxyl castor oil (Cremophor™), nonylphenol ethoxylate (Tergitol™), cyclodextrins, lecithin, methylbenzethonium chloride (Hyamine™), lithium dodecyl sulfate (LDS), sodium taurodeoxycholate (NaTDC), sodium taurocholate (NaTC), sodium glycocholate (NaGC), sodium deoxycholate (NaDC), sodium cholate, sodium alkylbenzene sulfonate (NaABS), N-lauroyl sarcosine (NLS), salts of carboxylicacids (i.e., Soaps), salts of sulfonic acids, salts of sulfuric acid, phosphoric and polyphosphoric acid esters, alkylphosphates, monoalkyl phosphate (MAP), and/or salts of perfluorocarboxylic acids.

In some embodiments, however, the composition can be substantially free or devoid of surfactant(s) or detergent(s) besides or other than SLS. In some embodiments, the composition can be substantially free or devoid of surfactant(s) or detergent(s) besides or other than CTAB. In some embodiments, the composition can be substantially free or devoid of surfactant(s) or detergent(s) besides or other than SLS and/or CTAB. In some embodiments, the composition can comprise SLS and be substantially free or devoid of surfactant(s) or detergent(s) besides or other than SLS. In some embodiments, the composition can comprise CTAB and be substantially free or devoid of surfactant(s) or detergent(s) besides or other than CTAB. In some embodiments, the composition can comprise SLS and/or CTAB and be substantially free or devoid of surfactant(s) or detergent(s) besides or other than SLS and/or CTAB.

In some embodiments, the composition can be substantially free or devoid of sodium dodecyl sulfate (SDS), urea, perchlorate, polysorbates (Tween™), lauryl dimethyl amine oxide, polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, Bile salts (sodium deoxycholate, sodium cholate), polyoxyl castor oil (Cremophor™), nonylphenol ethoxylate (Tergitol™), cyclodextrins, lecithin, methylbenzethonium chloride (Hyamine™), lithium dodecyl sulfate (LDS), sodium taurodeoxycholate (NaTDC), sodium taurocholate (NaTC), sodium glycocholate (NaGC), sodium deoxycholate (NaDC), sodium cholate, sodium alkylbenzene sulfonate (NaABS), N-lauroyl sarcosine (NLS), salts of carboxylicacids (i.e., Soaps), salts of sulfonic acids, salts of sulfuric acid, phosphoric and polyphosphoric acid esters, alkylphosphates, monoalkyl phosphate (MAP), and/or salts of perfluorocarboxylic acids. In some embodiments, the composition can be substantially free or devoid of SLS. In some embodiments, the composition can be substantially free or devoid of CTAB.

In some embodiments, the surfactant can be in, have, comprise, or be provided in a dry, solid, powdered, anhydrous, and/or granular form. In some embodiments, the surfactant can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the surfactant can comprise or be (provided) in the form of a stock solution (e.g., in water) having any suitable concentration (e.g., about 10%, 15%, 20%, 25%, 28%, 29%, 30%, 32%, 35%, 40%, or 45%, w/w, aqueous solution (e.g., in water), preferably about 30%, w/w, or molar equivalent thereof.

In some embodiments, the surfactant (e.g., SLS) can be included in the composition at about 2.99%, w/w, or 102 mM. In some embodiments, the surfactant can be included in the composition in a range of about 1% to about 5%, w/w, preferably about 1.5% to about 4.5%, w/w, more preferably about 2% to about 4%, w/w, still more preferably about 2.5% to about 3.5%, w/w, or a molar equivalent of any of the foregoing. In at least one embodiment, the composition can be (substantially) free or devoid of a surfactant or detergent besides SLS.

Without being bound to any theory, a surfactant or detergent may be useful to lyse cells, including contaminating microbes (e.g., bacteria), denature proteins, and allow release of nucleic acids.

### Alcohols

In at least one embodiment, the composition can include an alcohol (e.g., one or more alcohol). Preferably, the alcohol can be or comprise ethanol. More preferably, the alcohol can be or comprise a mixture of ethanol and one or more additional chemicals or components. In at least one embodiment, the one or more additional chemicals or components can be or comprise isopropanol. Still more preferably, the alcohol can be or comprise a mixture of ethanol and isopropanol. In at least one embodiment, the alcohol can be or comprise a specially denatured alcohol (SDA). More preferably, the alcohol can be or comprise SDA 3C, as known to those skilled in the art to comprise a mixture of about 95% ethanol v/v and about 5% isopropanol v/v. In at least one embodiment, the composition can include an alcohol consisting essentially of ethanol, or a mixture of ethanol and isopropanol. In at least one embodiment, the composition can include an alcohol consisting essentially of a specially denatured alcohol (SDA), preferably SDA 3C (specific gravity 0.79). Some embodiments can be alcohol-free.

In at least one alternative embodiment, the alcohol can be or comprise a mixture of ethanol and one or more additional chemicals or components selected from the group consisting of methanol, propanol, butanol, isobutanol, and so forth. The composition can include an alcohol, such as ethanol, methanol, propanol, and/or isopropanol, preferably a mixture of ethanol and another alcohol, such as methanol, n-propanol, isopropanol, n-butanol, trifluoroethanol, phenol, or 2,6-di-tert-butyl-4-methylphenol, more preferably a mixture of ethanol and isopropanol, still more preferably a specially denatured alcohol (SDA). In at least one alternative embodiment, the alcohol can be or comprise methanol, n-propanol, n-butanol, trifluoroethanol, phenol, or 2,6-di-tert-butyl-4-methylphenol.

In some embodiments, however, the composition can be substantially free or devoid of alcohol(s) besides or other than ethanol. In some embodiments, the composition can be substantially free or devoid of alcohol(s) besides or other than ethanol and isopropanol. In some embodiments, the composition can be substantially free or devoid of alcohol(s) besides or other than SDA 3C. In some embodiments, however, the composition can comprise ethanol and be substantially free or devoid of alcohol(s) besides or other than ethanol. In some embodiments, the composition can comprise ethanol and isopropanol and be substantially free or devoid of alcohol(s) besides or other than ethanol and isopropanol. In some embodiments, the composition can comprise SDA 3C and be substantially free or devoid of alcohol(s) besides or other than SDA 3C. In some embodiments, the composition can be substantially free or devoid of methanol, propanol, n-propanol, butanol, n-butanol, isobutanol, trifluoroethanol, phenol, and/or 2,6-di-tert-butyl-4-methylphenol.

In some embodiments, the alcohol can be in, have, comprise, or be provided in a liquid, aqueous, and/or solution form. In some embodiments, the alcohol can comprise or be (provided) in the form of a stock solution (e.g., in (RNAse-free) water) having any suitable concentration of alcohol (e.g., in the water). In some embodiments, the alcohol can be substantially pure, or a mixture of substantially pure alcohols. In some embodiments, the alcohol can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (or pure ethyl alcohol, 200 proof) (as measured by a suitable material assay).

In some embodiments, the alcohol can be or comprise a mixture or stock solution of or comprising about 95% v/v ethanol and about 5% v/v isopropanol. In some embodiments, the alcohol can be or comprise a mixture or stock solution of or comprising 90-99% v/v ethanol and about 1-10% v/v isopropanol. In certain embodiments, the alcohol can comprise a mixture of 50-99% ethanol v/v and 1-50% isopropanol v/v. More preferably, the alcohol can comprise a mixture of 60-98% ethanol v/v and 2-40% isopropanol v/v. Still more preferably, the alcohol can comprise a mixture of 75-97% ethanol v/v and 3-25% isopropanol v/v. Still more preferably, the alcohol can comprise a mixture of 80-96% ethanol v/v and 4-20% isopropanol v/v. Still more preferably, the alcohol can comprise a mixture of 85-95% ethanol v/v and 5-15% isopropanol v/v. Still more preferably, the alcohol can comprise a mixture of 90-95% ethanol v/v and 5-10% isopropanol v/v. Still more preferably, the alcohol can comprise a mixture of 92-95% ethanol v/v and 5-8% isopropanol v/v. Still more preferably, the alcohol can comprise a mixture of 95% ethanol v/v and 5% isopropanol v/v. Most preferably, the alcohol can be or comprise SDA 3C (specific gravity=0.79).

In some embodiments, the alcohol(s) (e.g., preferably comprising ethanol, ethanol and isopropanol, or SDA 3C) can be included in the composition in a range of about 1%, w/w, to about 30%, w/w, preferably about 2%, w/w, to about 28%, w/w, more preferably about 4%, w/w, to about 25%, w/w, still more preferably about 5%, w/w, to about 20%, w/w, or any value or range of values therebetween, or a molar equivalent of any of the foregoing. In some embodiments, the alcohol(s) can be included in the composition in a range of about 1%, w/w, to about 15%, w/w, or about 15%, w/w, to about 25%, w/w, preferably about 2%, w/w, to about 12%, w/w, or about 18%, w/w, to about 22%, w/w, more preferably about 5% to about 10%, w/w, or about 19%, w/w, to about 21%, w/w, still more preferably about 5%, w/w, or about 6%, w/w, about 10%, w/w (about 9.99%, w/w), or about 20%, w/w, (about 19.95%, w/w, or about 19.99%, w/w), or any value or range of values therebetween, or a molar equivalent of any of the foregoing.

In some embodiments, the amount of alcohol included in the composition can be less than about 20%, 21%, 22%, 23%, 24%, 25%, 26%, 27%, 28%, 29%, 30%, 32%, 35%, 38%, 40%, 45%, or 50%, w/w, or any value or range of values therebetween. In some embodiments, the amount of alcohol included in the composition can be less (e.g., about 5%, 10%, 15%, 20%, 22%, 25%, 30%, 35%, 40%, 45%, 50%, 55%, or 60% less) than typical, traditional, or existing nucleic acid or RNA preservation solutions (e.g., making the composition more amendable to shipping or transport).

Without being bound to any theory, alcohols may lyse cells, including contaminating microbes (e.g., bacteria) and/or denature proteins.

### Acids and pH

In at least one embodiment, the composition can include an acid (e.g., one or more acids). Preferably, the acid can be or comprise hydrochloric acid (HCl).

In at least one alternative embodiment, the acid can be or comprise hydrobromic acid (HBr), perchloric acid (HClO₄), nitric acid (HNO₃), or sulfuric acid (H₂SO₄). In at least one embodiment, the acid can be or comprise carbonic acid (H₂CO₃) or acetic acid (CH₃COOH). In at least one embodiment, the acid can be or comprise phosphoric acid (H₃PO₄), boric acid (H₃BO₃), or Emerald Safe acid (ESA), and so forth.

In some embodiments, however, the composition can be substantially free or devoid of acid(s) besides or other than HCl. In some embodiments, however, the composition can comprise HCl and be substantially free or devoid of acid(s) besides or other than HCl. In some embodiments, the composition can be substantially free or devoid of hydrobromic acid (HBr), perchloric acid (HClO₄), nitric acid (HNO₃), sulfuric acid (H₂SO₄), carbonic acid (H₂CO₃), acetic acid (CH₃COOH), phosphoric acid (H₃PO₄), boric acid (H₃BO₃), and/or Emerald Safe acid (ESA).

In some embodiments, the acid can be in, have, comprise, or be provided in a dry, solid, powdered, anhydrous, and/or granular form. In some embodiments, the acid can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the acid can comprise or be (provided) in the form of a concentrated stock solution (e.g., in water) having any suitable concentration (e.g., about 10%, 15%, 20%, 25%, 30%, 32%, 35%, 37%, 38%, 40%, 45%, 50%, or more, w/w, aqueous solution (e.g., in water), preferably about 37%, w/w, or molar equivalent thereof (e.g., between 1M and about 12M).

In at least one embodiment, the composition can have a pH in the range of about pH 4-7, pH 4.5-6.5, pH 4.5-6, pH 5-7, pH 5-6.5, or pH 5-6, (or any value or range of values therebetween). In some embodiments, the pH of the composition can be greater than 4. In some embodiments, the pH of the composition can be less than 7. In some embodiments, the pH of the composition can be greater than 4 and less than 7, preferably within a pH range of about 4.5 to about 6.5, more preferably within a pH range of about 5 to about 6, still more preferably within a pH range of about 5.2 to about 5.8, still more preferably within a pH range of about 5.3 to about 5.7, still more preferably within a pH range of about 5.4 to about 5.6, and most preferably, with a pH of about 5.5.

In at least one embodiment, an acid can be included in the composition q.s., or in a suitable amount to bring the composition to a pH in the range of about pH 4-7, pH 4.5-6.5, pH 4.5-6, pH 5-7, pH 5-6.5, or pH 5-6, (or any value or range of values therebetween), a pH greater than 4, a pH less than 7, a pH greater than 4 and less than 7, preferably within a pH range of about 4.5 to about 6.5, more preferably within a pH range of about 5 to about 6, still more preferably within a pH range of about 5.2 to about 5.8, still more preferably within a pH range of about 5.3 to about 5.7, still more preferably within a pH range of about 5.4 to about 5.6, and most preferably, with a pH of about 5.5.

Illustratively, in some embodiments, a suitable amount of a ˜37%, w/w, or ˜12M stock (aqueous) solution of HCl, or equivalent thereof, can be added or included in the composition q. s., or to bring the composition, to a pH in the range of about pH 4-7, pH 4.5-6.5, pH 4.5-6, pH 5-7, pH 5-6.5, or pH 5-6, (or any value or range of values therebetween), a pH greater than 4, a pH less than 7, a pH greater than 4 and less than 7, preferably within a pH range of about 4.5 to about 6.5, more preferably within a pH range of about 5 to about 6, still more preferably within a pH range of about 5.2 to about 5.8, still more preferably within a pH range of about 5.3 to about 5.7, still more preferably within a pH range of about 5.4 to about 5.6, and most preferably, with a pH of about 5.5.

Without being bound to any theory, acid(s) can be added to the composition to adjust (i.e., decrease) the pH of the composition. Adjusting the pH of the composition can affect the stability of RNA and/or other (macro)molecules in the sample.

Without being bound to any theory, it is noted, and those skilled in the art will appreciate that different acids have different “strengths” or the ability or tendency of the acid to lose a proton (H⁺). A strong acid is one that completely ionizes (dissociates) in a solution (provided there is sufficient solvent). In water, for example, one mole of a strong acid HA dissolves yielding one mole of H⁺ (as hydronium ion H₃O⁺ and higher aggregates) and one mole of the conjugate base, A. Essentially, none of the non-ionized acid HA remains. Some examples of strong acids are hydrochloric acid (HCl), hydroiodic acid (HI), hydrobromic acid (HBr), perchloric acid (HClO₄), nitric acid (HNO₃) and sulfuric acid (H₂SO₄). In aqueous solution, each of these essentially ionizes 100%. In contrast, a weak acid only partially dissociates. Examples in water include carbonic acid (H₂CO₃) and acetic acid (CH₃COOH). At equilibrium, both the acid and the conjugate base are present in solution. Stronger acids have a larger acid dissociation constant (Ka) and a smaller logarithmic constant (pKa=−log Ka) than weaker acids. The stronger an acid is, the more easily it loses a proton, H⁺. Two key factors that contribute to the ease of deprotonation are the polarity of the H-A bond and the size of atom A, which determines the strength of the

H-A bond. Acid strengths also depend on the stability of the conjugate base.

In light of the foregoing, the w/w amount of each acid necessary to bring the pH of the composition to a desired level is different. For instance, illustratively, while (about) 4% hydrochloric acid 37%, w/w (in water), may be sufficient to bring certain embodiments of the present disclosure to pH (about) 5.5, 4% acetic acid 37%, w/w (in water), may be too weak to bring a similar embodiment to pH (about) 5.5, 4% sulfuric acid 37%, w/w (in water), may be too strong to bring the embodiment to pH (about) 5.5, 4% nitric acid 37%, w/w (in water), may oxidize the alcohol, and so forth. Without being bound to any theory, even those of ordinary skill in the art may not, with further experimentation, be able to determine which acids are suitable in one or more embodiments of the present disclosure.

### Reducing Agents

In at least one embodiment, the composition can include a reducing agent (e.g., one or more reducing agents). Preferably, the reducing agent can be or comprise tris(2-carboxyethyl)phosphine hydrochloride (TCEP; MW 286.65) or DL-Dithiothreitol (DTT; MW 154.3), more preferably TCEP. In at least one embodiment, the composition can include a reducing agent consisting essentially of tris(2-carboxyethyl)phosphine hydrochloride (TCEP) or DL-Dithiothreitol (DTT), more preferably TCEP. In at least one embodiment, the composition can include a reducing agent consisting essentially of tris(2-carboxyethyl)phospine hydrochloride (TCEP). In at least one embodiment, the composition can include a reducing agent consisting essentially of DL-Dithiothreitol (DTT).

In some embodiments, TCEP can be preferable over DTT or other (stronger, more popular) reducing agents (e.g., acetylcysteine (e.g., N-acetylcysteine (NAC), including N-acetyl-L-cysteine, N-acetyl-D-cysteine, and racemic N-acetylcysteine or a (racemic) mixture of N-acetyl-L-cysteine and N-acetyl-D-cysteine), ascorbic acid, dithionite, erythiorbate, cysteine, glutathione, 2-mercaptoethanol (BME), dierythritol, a resin-supported thiol, a resin-supported phosphine, vitamin E, and/or trolox, or salts thereof, sodium citrate, potassium citrate, potassium iodide, ammonium chloride, guaiphenesin (or guaifenesin), Tolu balsam, Vasaka, ambroxol, carbocisteine, erdosteine, mecysteine, dornase alfa, and so forth. Without being bound to any theory, some (strong) reducing agents can have an often unpleasant, “rotten-egg” smell. In some embodiments, an unpleasant smell may be more undesirable than a weaker reducing agent. Illustratively, in some saliva collection devices, kits, and/or systems, users may bring their nose close enough to the solution-containing (collection) container to smell the reducing agent.

In at least one alternative embodiment, however, the reducing agent can be or comprise an acetylcysteine (e.g., N-acetylcysteine (NAC), including N-acetyl-L-cysteine, N-acetyl-D-cysteine, and racemic N-acetylcysteine or a (racemic) mixture of N-acetyl-L-cysteine and N-acetyl-D-cysteine), ascorbic acid, dithionite, erythiorbate, cysteine, mecysteine, carbocisteine glutathione, dithiothreitol (DTT), 2-mercaptoethanol (BME), dierythritol, a resin-supported thiol, a resin-supported phosphine, vitamin E, and/or trolox, or salts thereof.

In one or more embodiments, the composition does not contain an acetylcysteine, N-acetylcysteine (NAC), N-acetyl-L-cysteine, N-acetyl-D-cysteine, racemic N-acetylcysteine or a (racemic) mixture of N-acetyl-L-cysteine and N-acetyl-D-cysteine, ascorbic acid, dithionite, erythiorbate, dithiothreitol (DTT), 2-mercaptoethanol (BME), dierythritol, a resin-supported thiol, a resin-supported phosphine, vitamin E, trolox, and/or salts thereof. At least one embodiment is (substantially) devoid of an acetylcysteine, N-acetylcysteine (NAC), N-acetyl-L-cysteine, N-acetyl-D-cysteine, racemic N-acetylcysteine or a (racemic) mixture of N-acetyl-L-cysteine and N-acetyl-D-cysteine, ascorbic acid, dithionite, erythiorbate, dithiothreitol, 2-mercaptoethanol, dierythritol, a resin-supported thiol, a resin-supported phosphine, vitamin E, trolox, and/or salts thereof. In at least one embodiment, the composition can be (substantially) free or devoid of DTT, BME, N-acetyl-D-cysteine, and/or any of the other foregoing reducing agents (e.g., N-acetyl-L-cysteine). In at least one embodiment, the composition can be (substantially) free or devoid of a reducing agent besides (or other than) TCEP. In some embodiments, the composition can include TCEP and be (substantially) free or devoid of a reducing agent besides (or other than) TCEP.

In some embodiments, the reducing agent can be in, have, comprise, or be provided in a dry, solid, powdered, anhydrous, and/or granular form. In some embodiments, the reducing agent can have a purity of at least, up to, and/or about 90%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the reducing agent can comprise or be (provided) in the form of a stock solution (e.g., in water) having any suitable concentration.

The reducing agent (e.g., TCEP) can be included in the composition in a range of about 0.01% to about 1%, preferably about 0.05% to about 0.5%, more preferably about 0.08% to about 0.4%, still more preferably about 0.1% to about 0.3%, still more preferably about 0.12% to about 0.25%, still more preferably about 0.15% to about 0.22%, w/w, still more preferably about 0.17% to about 0.2%, w/w, still more preferably about 0.17%, w/w, or about 0.2%, w/w.

Without being bound to any theory, reducing agents can be or comprise a mucolytic agents and/or may aid in denaturing proteins (e.g., by reducing or cleaving disulfide bridges). In addition, ingredients or components (e.g., chemicals or agents) containing free sulfhydryl groups may act as antioxidants and/or may help control dissolved oxygen in the RNA Stabilizing Solution. In some embodiments, a mucolytic agent that is not a reducing agent (for the reduction of cysteine bonds) can be used, either in addition to or in the place of the reducing agent.

### Optional Visual Indicators

Some embodiments can include a visual indicator. The visual indicator can be or comprise a coloring agent. The visual indicator can be or comprise a dye or colored dye. The dye or colored dye can be or comprise a blue dye. The blue dye can be or comprise FD&C Blue No. 1 (e.g., Erioglaucine). Thus, in some embodiments, the composition can include a visual indicator, preferably a coloring agent, more preferably a colored dye, still more preferably a blue dye, still more preferably FD&C Blue No. 1.

In some embodiments, the visual indicator can be in, have, comprise, or be provided in a dry, solid, powdered, anhydrous, and/or granular form. In some embodiments, the visual indicator can have a purity of at least, up to, and/or about 80%, 85%, 86%, 87%, 88%, 89%, 90%, 91%, 92%, 93%, 94%, 95%, 96%, 97%, 98%, 99%, 99.1%, 99.2%. 99.3%, 99.4%, 99.5%, 99.6%, 99.7%, 99.8%, or 99.9% (as measured by a suitable material assay). In some embodiments, the visual indicator can comprise or be (provided) in the form of a stock (solution (e.g., in water)) having any suitable concentration (e.g., about 0.01%, 0.05%, 0.075%, 0.1%, 0.125%, 0.15%, 0.175%, 0.2%, 0.25%, 0.3%, or 0.5%, w/w, aqueous solution (e.g., in water), preferably a 0.2% concentrate. In some embodiments, stock solution can be made using the dry, solid, powdered, anhydrous, and/or granular material.

The visual indicator (e.g., FD&C Blue No. 1) can be included in the composition in any visually suitable amount, such as in a range of about 0.00005% to about 0.001%, preferably about 0.0001% to about 0.00075%, more preferably about 0.0002% to about 0.0005%, w/w, still more preferably about 0.0003% to about 0.00045%, w/w, still more preferably about 0.00035% to about 0.0004%, w/w, still more preferably about 0.00037%, w/w, or about 0.0004%, w/w.

In at least one embodiment, the visual indicator (e.g., FD&C Blue No. 1) can be added to the composition as a concentrate, such as a 0.2% concentrate. The concentrate can be an aqueous or water-based concentrate in some embodiments. In some embodiments, the composition can include about 0.01-2.5%, w/w, of a 0.01-5%, w/w (in water) visual indicator concentrate, preferably about 0.05-1%, w/w, of a 0.05-1%, w/w (in water) visual indicator concentrate, more preferably about 0.075-0.5%, w/w, of a 0.075-0.5%, w/w (in water) visual indicator concentrate, still more preferably about 0.1-0.25%, w/w, of a 0.1-0.25%, w/w (in water) visual indicator concentrate, still more preferably about 0.185% w/w of 0.2% w/w (in water) visual indicator concentrate. In at least one embodiment, the visual indicator (e.g., FD&C Blue No. 1) can be included in the composition at about 0.185%, w/w, of a ˜0.2% stock (aqueous) solution, or equivalent thereof. In some embodiments, the visual indicator (e.g., FD&C Blue No. 1) can be included in the composition at about 0.6%, w/w, of a about 0.2%, w/w, concentrate or stock (aqueous) solution, or equivalent thereof.

### Antimicrobials

In some embodiments, the composition can include an antimicrobial agent. In some embodiments, one or more of the foregoing components can exhibit antimicrobial activity. For instance, the alcohol, chaotropic agent, surfactant, and/or reducing agent can be antimicrobial or exhibit antimicrobial activity in some embodiments. Accordingly, certain embodiments need not include a separate antimicrobial (e.g., bactericidal and/or bacteriostatic) agent. In one or more embodiments, the antimicrobial properties of alcohol (e.g., SDA 3C) persist even at the lower concentrations in which the alcohol is provided in said embodiment(s) of the present disclosure (e.g., about 9.99%-19.99%, w/w, etc.). In some embodiments, the composition can be substantially free or devoid of antimicrobial agent(s), bactericidal agent(s), and/or bacteriostatic agent(s) other than the chaotropic agent, the surfactant, the alcohol, and the reducing agent.

### Ribonuclease Inhibitors

Some embodiments include a ribonuclease inhibitor, or inhibitor of ribonuclease, such as heparin, heparan sulfate, oligo (vinylsulfonic acid), poly(vinylsulfonic acid), oligo(vinylphosphonic acid), and poly(vinylsulfonic acid), or salts thereof. In certain (e.g., preferred) embodiments, the composition does not include a ribonuclease inhibitor or inhibitor of ribonuclease, or is (substantially) free or devoid of one or more (e.g., any) ribonuclease inhibitor or inhibitor of ribonuclease (e.g., other than the chaotropic agent, such as LiCl, which may have intrinsic RNAse inhibitory activity, the surfactant, the alcohol, and the reducing agent).

### Proteases

Some embodiments include a protease. In certain (e.g., preferred) embodiments, the composition does not include a protease, or is (substantially) free or devoid of one or more (e.g., any) protease(s). In some embodiments, the composition does not include, or is (substantially) free or devoid of proteinase K. Without being bound to any theory, a protease (or proteolytic enzyme, peptidase or proteinase) is a type of enzyme that breaks one or more peptide bonds through hydrolysis, thereby converting proteins into smaller protein fragments (or peptides) or individual protein subunits (or amino acids).

### Protein Denaturants

Some embodiments include one or more protein denaturants. For instance, in at least one embodiment, the (i) chaotropic agent can be, comprise, or function as a protein denaturant (or denature proteins or have or exhibit protein denaturation activity). In at least one embodiment, the (ii) surfactant/detergent can be, comprise, or function as a protein denaturant (or denature proteins or have or exhibit protein denaturation activity). In at least one embodiment, the (iii) alcohol can be, comprise, or function as a protein denaturant (or denature proteins or have or exhibit protein denaturation activity). In at least one embodiment, the (iv) reducing agent can be, comprise, or function as a protein denaturant (or denature proteins or have or exhibit protein denaturation activity), such as when the protein(s) contain accessible disulfide bonds or bridges. In some embodiments, two or more of the (i) chaotropic agent, (ii) surfactant/detergent, (iii) alcohol, and (iv) reducing agent can be, comprise, or function as a protein denaturant (or denature proteins or have or exhibit protein denaturation activity). In some embodiments, each or all of the (i) chaotropic agent, (ii) surfactant/detergent, (iii) alcohol, and (iv) reducing agent can be, comprise, or function as a protein denaturant (or denature proteins or have or exhibit protein denaturation activity).

Without being bound to any theory, the protein denaturation activity of one or more of the foregoing components or ingredients can be concentration and/or time dependent.

### Formulations

Various embodiments of the present disclosure include a nucleic acid (e.g., RNA) preservation composition (or formulation), comprising a carrier, a chaotropic agent, a buffering agent, a chelating agent, a surfactant, an alcohol, and/or a reducing agent. Some embodiments further include an optional acid or base, to bring the composition to a preferred pH or pH range. Some embodiments further include an optional visual indicator.

An embodiment of the present disclosure includes a ribonucleic acid (RNA) preservation composition, comprising a carrier, a buffer or buffering agent, and a (metal) chelating agent. Another embodiment of the present disclosure includes an RNA preservation composition, comprising a carrier, a buffering agent, a chelating agent, and a chaotropic agent. Another embodiment of the present disclosure includes an RNA preservation composition, comprising a carrier, a buffering agent, a chelating agent, a chaotropic agent, and a detergent or a surfactant. Another embodiment of the present disclosure includes an RNA preservation composition, comprising a carrier, a buffering agent, a chelating agent, a chaotropic agent, and an alcohol. Another embodiment of the present disclosure includes an RNA preservation composition, comprising a carrier, a buffering agent, a chelating agent, a chaotropic agent, and a reducing agent. Another embodiment of the present disclosure includes an RNA preservation composition, comprising a carrier, a buffering agent, a chelating agent, a chaotropic agent, a reducing agent, and an alcohol. Another embodiment of the present disclosure includes an RNA preservation composition, comprising a carrier, a buffering agent, a chelating agent, a chaotropic agent, a reducing agent, and a detergent (or surfactant). Another embodiment of the present disclosure includes an RNA preservation composition, comprising an aqueous carrier, a chaotropic agent, a buffering agent, a (metal) chelating agent, a detergent (or surfactant), an alcohol, and a reducing agent.

In some embodiments, an acid (or base) can be added to achieve a suitable final pH. The composition can have a pH of 4-7 and/or an acid q.s. to a pH of 4-7. Preferably, the composition can have a pH of about 5.5 and/or an acid q.s. to a pH of about 5.5. The composition can also include an optional visual indicator.

An embodiment can include, for example, 1-10% chaotropic agent, w/w, 1-10% buffering agent, w/w, 0.01-2% chelating agent, w/w, 1-5% surfactant, w/w, 5-40% alcohol, w/w, and/or 0.01-0.2% reducing agent, w/w. The composition can have pH 4-7, or an acid qs to pH 4-7. The composition can have a carrier qs to 100%. An embodiment can further include 0.005-2.5%, w/w, visual indicator.

An embodiment can included, for example, about 3%-7%, w/w, of the chaotropic agent, about 4-6%, w/w, of the buffering agent, about 0.1-0.3%, w/w, of the chelating agent, about 2%-4%, w/w, of the surfactant, about 9%-21%, w/w, of the alcohol, and/or about 0.1-0.25%, w/w, of the reducing agent.

An embodiment can included, for example, about 3.33%-6.65%, w/w, of the chaotropic agent, about 5.99%, w/w, of the buffering agent, about 0.2%, w/w, of the chelating agent, about 2.99%, w/w, of the surfactant, about 9.98%-19.96%, w/w, of the alcohol, and/or about 0.17%, w/w, of the reducing agent.

In some embodiments, the composition can be substantially free or devoid of microbial (e.g., bacterial, fungal, and/or viral) contamination. In some embodiments, the composition can have less than or equal to (about) 100 cfu/g bacteria or bacterial contamination. In some embodiments, the composition can have less than or equal to (about) 99, 98, 97, 96, 95, 90, 85, 80, 75, 70, 65, 60, 55, 50, 45, 40, 35, 30, 25, 20, 15, 10, or 5 cfu/g bacteria or bacterial contamination. In some embodiments, the composition can have less than or equal to (about) 100 cfu/g fungus (or fungi, such as yeast and/or mold) or fungal contamination. In some embodiments, the composition can have less than or equal to (about) 99, 98, 97, 96, 95, 90, 85, 80, 75, 70, 65, 60, 55, 50, 45, 40, 35, 30, 25, 20, 15, 10, or 5 cfu/g fungus (or fungi, such as yeast and/or mold) or fungal contamination. As used herein, “cfu/g” refers to colony forming units (of the one or more microbes) per gram (of the (final and/or liquid) composition).

The Tables below present various exemplary, non-limiting embodiments of compositions in accordance with the present disclosure.

TABLE 1a

| Ingredients for the preparation of ~150 mL of RNA | Solution 1.0 | |
|---|---|---|
| Preserving Solution 1.0 | Amount (g) | % w/w |
| 1. RNAse-free Water (qs to Final Volume of 150 mL) | ~137 | ~91.82% |
| 2. Lithium Chloride (LiCl) | 5.00 | 3.33% |
| 3. Edetate Disodium Dihydrate (EDTA•2H2O) | 4.00 | 2.66% |
| 4. Trisodium Citrate Dihydrate (C6H5O7Na3•2H2O) | 0.15 | 0.10% |
| 6. N-Lauroylsarcosine sodium salt (Sarkosyl detergent) | 3.00 | 2.00% |
| 7. Concentrated Hydrochloric Acid (HCl) to pH = 5.5 | q.s. | |
| 8. DL-Dithiothreitol (DTT) | 0.15 | 0.10% |
| TOTAL | ~150 | 100% |

TABLE 1b

| Ingredients for the preparation of ~300 mL of RNA | Solution 1.0 | |
|---|---|---|
| Preserving Solution 1.0 | Amount (g) | % w/w |
| 1. RNAse-free Water (qs to Final Volume of 300 mL) | ~275 | ~91.82% |
| 2. Lithium Chloride (LiCl) | 10.00 | 3.33% |
| 3. Edetate Disodium Dihydrate (EDTA•2H2O) | 8.00 | 2.66% |
| 4. Trisodium Citrate Dihydrate (C6H5O7Na3•2H2O) | 0.30 | 0.10% |
| 6. N-Lauroylsarcosine sodium salt (Sarkosyl detergent) | 6.00 | 2.00% |
| 7. Concentrated Hydrochloric Acid (HCl) to pH = 5.5 | q.s. | |
| 8. DL-Dithiothreitol (DTT) | 0.30 | 0.10% |
| TOTAL | ~300 | 100% |

TABLE 2

| Ingredients for the preparation of ~150 mL of RNA | Solution 1.1 | |
|---|---|---|
| Preserving Solution 1.1 | Amount (g) | % w/w |
| 1. RNAse-free Water (qs to Final Volume of 150 mL) | ~115 | ~77.31% |
| 2. Lithium Chloride (LiCl) | 5.00 | 3.33% |
| 3. Edetate Disodium Dihydrate (EDTA•2H2O) | 0.30 | 0.20% |
| 4. Trisodium Citrate Dihydrate (C6H5O7Na3•2H2O) | 9.00 | 6.00% |
| 5. SDA 3C Alcohol (specific gravity = 0.79) | 15.00 | 10.00% |
| 6. N-Lauroylsarcosine sodium salt (Sarkosyl detergent) | 4.50 | 3.00% |
| 7. Concentrated Hydrochloric Acid (HCl) to pH = 5.5 | q.s. | |
| 8. Tris(2-carboxyethyl)phosphine hydrochloride (TCEP) | 0.25 | 0.17% |
| TOTAL | ~150 | 100% |

TABLE 3

| Ingredients for the preparation of ~300 mL of RNA | Solution 1.1a | |
|---|---|---|
| Preserving Solution 1.1a | Amount (g) | % w/w |
| 1. RNAse-free Water (q.s. to Final Volume of 300 mL) | ~241 | ~80.61% |
| 2. Lithium Chloride (LiCl) | 10.00 | 3.33% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 30.00 | 9.99% |
| 6. N-Lauroylsarcosine sodium salt (Sarkosyl detergent) | 12.00 | 4.00% |
| 7. Concentrated Hydrochloric Acid (HCl) to pH = 5.5 | q.s. | |
| 8. Tris(2-carboxyethyl)phosphine hydrochloride (TCEP) | 0.60 | 0.20% |
| TOTAL | ~300 | 100% |

TABLE 4

| Ingredients for the preparation of ~150 mL of RNA | Solution 1.1b | |
|---|---|---|
| Preserving Solution 1.1b | Amount (g) | % w/w |
| 1. RNAse-free Water (q.s. to Final Volume of 150 mL) | ~110 | ~73.98% |
| 2. Lithium Chloride (LiCl) | 10.00 | 6.66% |
| 3. Edetate Disodium Dihydrate (EDTA•2H2O) | 0.30 | 0.20% |
| 4. Trisodium Citrate Dihydrate (C6H5O7Na3•2H2O) | 9.00 | 6.00% |
| 5. SDA 3C Alcohol (specific gravity = 0.79) | 15.00 | 10.00% |
| 6. N-Lauroylsarcosine sodium salt (Sarkosyl detergent) | 4.50 | 3.00% |
| 7. Concentrated Hydrochloric Acid (HCl) to pH = 5.5 | q.s. | |
| 8. Tris(2-carboxyethyl)phosphine hydrochloride (TCEP) | 0.25 | 0.17% |
| TOTAL | ~150 | 100% |

TABLE 5

| Ingredients for the preparation of ~150 mL of RNA | Solution 1.1bc | |
|---|---|---|
| Preserving Solution 1.1bc | Amount (g) | % w/w |
| 1. RNAse-free Water (q.s. to Final Volume of 150 mL) | ~100 | ~67.31% |
| 2. Lithium Chloride (LiCl) | 5.00 | 3.33% |
| 3. Edetate Disodium Dihydrate (EDTA•2H2O) | 0.30 | 0.20% |
| 4. Trisodium Citrate Dihydrate (C6H5O7Na3•2H2O) | 9.00 | 6.00% |
| 5. SDA 3C Alcohol (specific gravity = 0.79) | 30.00 | 19.99% |
| 6. N-Lauroylsarcosine sodium salt (Sarkosyl detergent) | 4.50 | 3.00% |
| 7. Concentrated Hydrochloric Acid (HCl) to pH = 5.5 | q.s. | |
| 8. Tris(2-carboxyethyl)phosphine hydrochloride (TCEP) | 0.25 | 0.17% |
| TOTAL | ~150 | 100% |

TABLE 6

| Ingredients for the preparation of ~300 mL of RNA | Solution 1.1c | |
|---|---|---|
| Preserving Solution 1.1c | Amount (g) | % w/w |
| 1. RNAse-free Water (q.s. to Final Volume of 300 mL) | ~256 | ~85.61% |
| 2. Lithium Chloride (LiCl) | 10.00 | 3.33% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 15.00 | 5.00% |
| 6. N-Lauroylsarcosine sodium salt (Sarkosyl detergent) | 12.00 | 4.00% |
| 7. Concentrated Hydrochloric Acid (HCl) to pH = 5.5 | q.s. | |
| 8. Tris(2-carboxyethyl)phosphine hydrochloride (TCEP) | 0.60 | 0.20% |
| TOTAL | ~300 | 100% |

TABLE 7

| Ingredients for the preparation of ~150 mL of RNA | Solution 1.1d | |
|---|---|---|
| Preserving Solution 1.1d | Amount (g) | % w/w |
| 1. RNAse-free Water (q.s. to Final Volume of 150 mL) | ~95 | ~63.98% |
| 2. Lithium Chloride (LiCl) | 10.00 | 6.66% |
| 3. Edetate Disodium Dihydrate (EDTA•2H2O) | 0.30 | 0.20% |
| 4. Trisodium Citrate Dihydrate (C6H5O7Na3•2H2O) | 9.00 | 6.00% |
| 5. SDA 3C Alcohol (specific gravity = 0.79) | 30.00 | 19.99% |
| 6. N-Lauroylsarcosine sodium salt (Sarkosyl detergent) | 4.50 | 3.00% |
| 7. Concentrated Hydrochloric Acid (HCl) to pH = 5.5 | q.s | |
| 8. Tris(2-carboxyethyl)phosphine hydrochloride (TCEP) | 0.25 | 0.17% |
| TOTAL | ~150 | 100% |

TABLE 8

| Ingredients for the preparation of ~300 mL of RNA | Solution 1.1e | |
|---|---|---|
| Preserving Solution 1.1e | Amount (g) | % w/w |
| 1. RNAse-free Water (q.s. to Final Volume of 300 mL) | ~241 | ~80.61% |
| 2. Lithium Chloride (LiCl) | 10.00 | 3.33% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 30.00 | 9.99% |
| 6. Cetyltrimethylammonium bromide (CTAB) | 12.00 | 4.00% |
| 7. Concentrated Hydrochloric Acid (HCl) to pH = 5.5 | q.s | |
| 8. Tris(2-carboxyethyl)phosphine hydrochloride (TCEP) | 0.60 | 0.20% |
| TOTAL | ~300 | 100% |

TABLE 9

| Ingredients for the preparation of ~150 mL of RNA | Solution 1.1ef | |
|---|---|---|
| Preserving Solution 1.1ef | Amount (g) | % w/w |
| 1. RNAse-free Water (q.s. to Final Volume of 150 mL) | ~95 | ~63.98% |
| 2. Lithium Chloride (LiCl) | 10.00 | 6.66% |
| 3. Edetate Disodium Dihydrate (EDTA•2H2O) | 0.30 | 0.20% |
| 4. Trisodium Citrate Dihydrate (C6H5O7Na3•2H2O) | 9.00 | 6.00% |
| 5. SDA 3C Alcohol (specific gravity = 0.79) | 30.00 | 19.99% |
| 6. Cetyltrimethylammonium bromide (CTAB) | 4.50 | 3.00% |
| 7. Concentrated Hydrochloric Acid (HCl) to pH = 5.5 | q.s | |
| 8. Tris(2-carboxyethyl)phosphine hydrochloride (TCEP) | 0.25 | 0.17% |
| TOTAL | ~150 | 100% |

TABLE 10

| Ingredients for the preparation of ~300 mL of RNA | Solution 1.1f | |
|---|---|---|
| Preserving Solution 1.1f | Amount (g) | % w/w |
| 1. RNAse-free Water (q.s. to Final Volume of 300 mL) | ~231 | ~77.28% |
| 2. Lithium Chloride (LiCl) | 20.00 | 6.66% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 30.00 | 9.99% |
| 6. Cetyltrimethylammonium bromide (CTAB) | 12.00 | 4.00% |
| 7. Concentrated Hydrochloric Acid (HCl) to pH = 5.5 | q.s | |
| 8. Tris(2-carboxyethyl)phosphine hydrochloride (TCEP) | 0.60 | 0.20% |
| TOTAL | ~300 | 100% |

TABLE 11

| Ingredients for the preparation of ~300 mL of RNA | Solution 1.1g | |
|---|---|---|
| Preserving Solution 1.1g | Amount (g) | % w/w |
| 1. RNAse-free Water (q.s. to Final Volume of 300 mL) | ~256 | ~85.61% |
| 2. Lithium Chloride (LiCl) | 10.00 | 3.33% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 15.00 | 5.00% |
| 6. Cetyltrimethylammonium bromide (CTAB) | 12.00 | 4.00% |
| 7. Concentrated Hydrochloric Acid (HCl) to pH = 5.5 | q.s | |
| 8. Tris(2-carboxyethyl)phosphine hydrochloride (TCEP) | 0.60 | 0.20% |
| TOTAL | ~300 | 100% |

TABLE 12

| Ingredients for the preparation of ~300 mL of RNA | Solution 1.1h | |
|---|---|---|
| Preserving Solution 1.1h | Amount (g) | % w/w |
| 1. RNAse-free Water (q.s. to Final Volume of 300 mL) | ~246 | ~82.28% |
| 2. Lithium Chloride (LiCl) | 20.00 | 6.66% |
| 3. Edetate Disodium Dihydrate (EDTA•2H2O) | 0.60 | 0.20% |
| 4. Trisodium Citrate Dihydrate (C6H5O7Na3•2H2O) | 5.00 | 1.67% |
| 5. SDA 3C Alcohol (specific gravity = 0.79) | 15.00 | 5.00% |
| 6. Cetyltrimethylammonium bromide (CTAB) | 12.00 | 4.00% |
| 7. Concentrated Hydrochloric Acid (HCl) to pH = 5.5 | q.s | |
| 8. Tris(2-carboxyethyl)phosphine hydrochloride (TCEP) | 0.60 | 0.20% |
| TOTAL | ~300 | 100% |

TABLE 13

| Ingredients for the preparation of ~300 mL of RNA | Solution 1.1i | |
|---|---|---|
| Preserving Solution 1.1i | Amount (g) | % w/w |
| 1. RNAse-free Water (q.s. to Final Volume of 150 mL) | ~264 | ~88.22 |
| 2. Lithium Chloride (LiCl) | 15.00 | 4.99% |
| 3. Edetate Disodium Dihydrate (EDTA•2H2O) | 1.50 | 0.50% |
| 4. Trisodium Citrate Dihydrate (C6H5O7Na3•2H2O) | 6.00 | 2.00% |
| 5. SDA 3C Alcohol (specific gravity = 0.79) | 0 | 0% |
| 6. N-Lauroylsarcosine sodium salt (Sarkosyl detergent) | 12.00 | 3.99% |
| 7. Concentrated Hydrochloric Acid (HCl) to pH = 5.5 | q.s | |
| 8. Tris(2-carboxyethyl)phosphine hydrochloride (TCEP) | 0.90 | 0.30% |
| TOTAL | ~300 | 100% |

TABLE 14

| Ingredients for the preparation of ~300 mL of RNA | Solution 1.1j | |
|---|---|---|
| Preserving Solution 1.1j | Amount (g) | % w/w |
| 1. RNAse-free Water (q.s. to Final Volume of 150 mL) | ~246 | ~82.23 |
| 2. Lithium Chloride (LiCl) | 15.00 | 4.99% |
| 3. Edetate Disodium Dihydrate (EDTA•2H2O) | 1.50 | 0.50% |
| 4. Trisodium Citrate Dihydrate (C6H5O7Na3•2H2O) | 6.00 | 2.00% |
| 5. SDA 3C Alcohol (specific gravity = 0.79) | 18.00 | 5.99% |
| 6. N-Lauroylsarcosine sodium salt (Sarkosyl detergent) | 12.00 | 3.99% |
| 7. Concentrated Hydrochloric Acid (HCl) to pH = 5.5 | q.s | |
| 8. Tris(2-carboxyethyl)phosphine hydrochloride (TCEP) | 0.90 | 0.30% |
| TOTAL | ~300 | 100% |

TABLE 15

| Ingredients for the preparation of ~300 mL of RNA | Solution 1.1k | |
|---|---|---|
| Preserving Solution 1.1k | Amount (g) | % w/w |
| 1. RNAse-free Water (q.s. to Final Volume of 150 mL) | ~264 | ~88.22 |
| 2. Lithium Chloride (LiCl) | 15.00 | 4.99% |
| 3. Edetate Disodium Dihydrate (EDTA•2H2O) | 1.50 | 0.50% |
| 4. Trisodium Citrate Dihydrate (C6H5O7Na3•2H2O) | 6.00 | 2.00% |
| 5. SDA 3C Alcohol (specific gravity = 0.79) | 0 | 0% |
| 6. Cetyltrimethylammonium bromide (CTAB) | 12.00 | 3.99% |
| 7. Concentrated Hydrochloric Acid (HCl) to pH = 5.5 | q.s | |
| 8. Tris(2-carboxyethyl)phosphine hydrochloride (TCEP) | 0.90 | 0.30% |
| TOTAL | ~300 | 100% |

TABLE 16

| Ingredients for the preparation of ~300 mL of RNA | Solution 1.1l | |
|---|---|---|
| Preserving Solution 1.1l | Amount (g) | % w/w |
| 1. RNAse-free Water (q.s. to Final Volume of 150 mL) | ~246 | ~82.23 |
| 2. Lithium Chloride (LiCl) | 15.00 | 4.99% |
| 3. Edetate Disodium Dihydrate (EDTA•2H2O) | 1.50 | 0.50% |
| 4. Trisodium Citrate Dihydrate (C6H5O7Na3•2H2O) | 6.00 | 2.00% |
| 5. SDA 3C Alcohol (specific gravity = 0.79) | 18.00 | 5.99% |
| 6. Cetyltrimethylammonium bromide (CTAB) | 12.00 | 3.99% |
| 7. Concentrated Hydrochloric Acid (HCl) to pH = 5.5 | q.s | |
| 8. Tris(2-carboxyethyl)phosphine hydrochloride (TCEP) | 0.90 | 0.30% |
| TOTAL | ~300 | 100% |

To any of the foregoing formulations, or other embodiments of the present disclosure, a suitable amount of a visual indicator can be added (e.g., 0.2%, w/w, of a 0.2% concentrate of FD&C Blue No.1 (Erioglaucine).

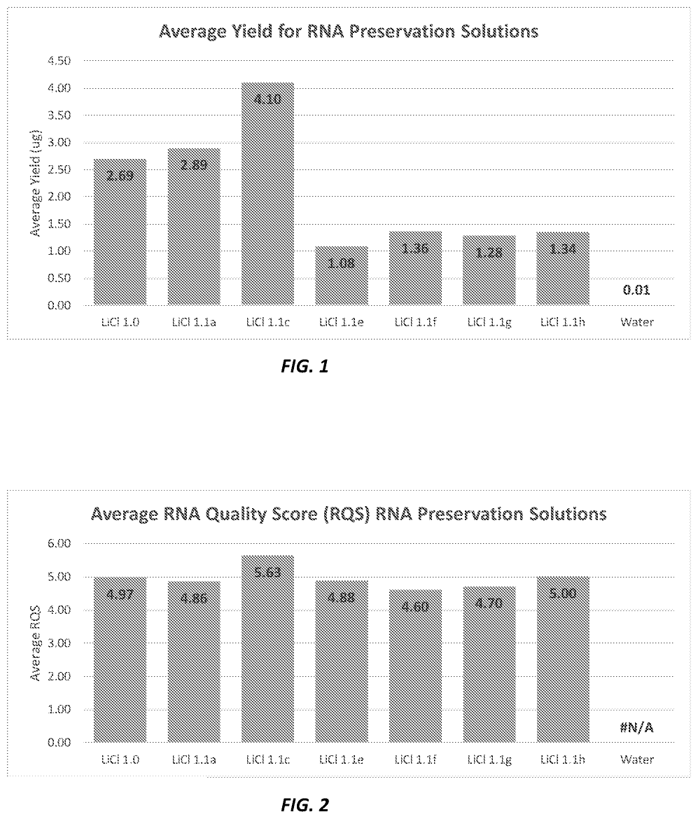

FIGS. 1 and 2 present RNA yield (FIG. 1) and RNA Quality Score data (FIG. 2) for various specific formulations of the composition of the present disclosure. In each example, 1.5 mL of the indicated RNA preserving solution was added to 1.5 mL of (human) saliva. Preserved sample testing occurred after 5 days at room temperature and after 7 days with refrigeration. It will be appreciated that each of the specific formulations of the composition of the present disclosure yields a sufficient amount and quality of RNA for subsequent analysis. Longer preservation testing results (not shown) indicate that the inventive compositions stabilize RNA in solution from human saliva samples well beyond 5 days at room temperature and 7 days with refrigeration.

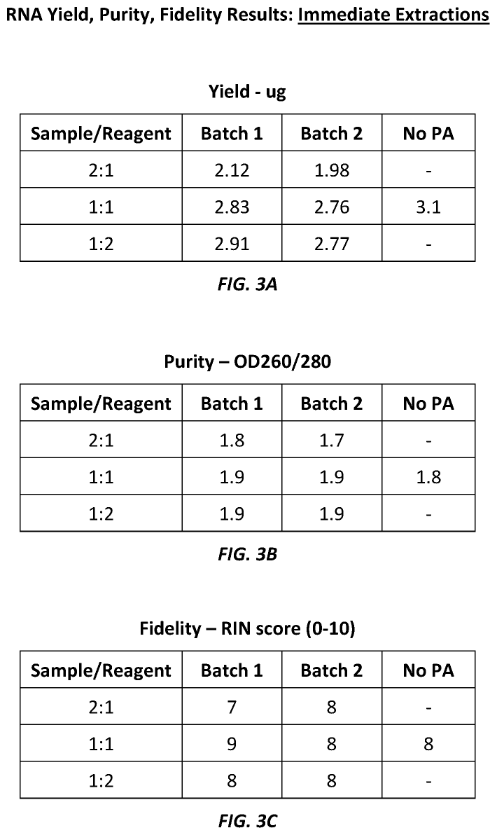

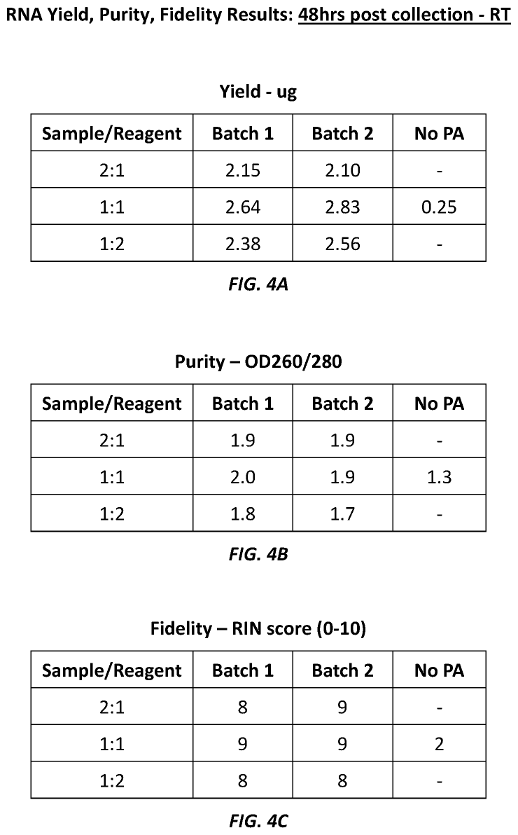

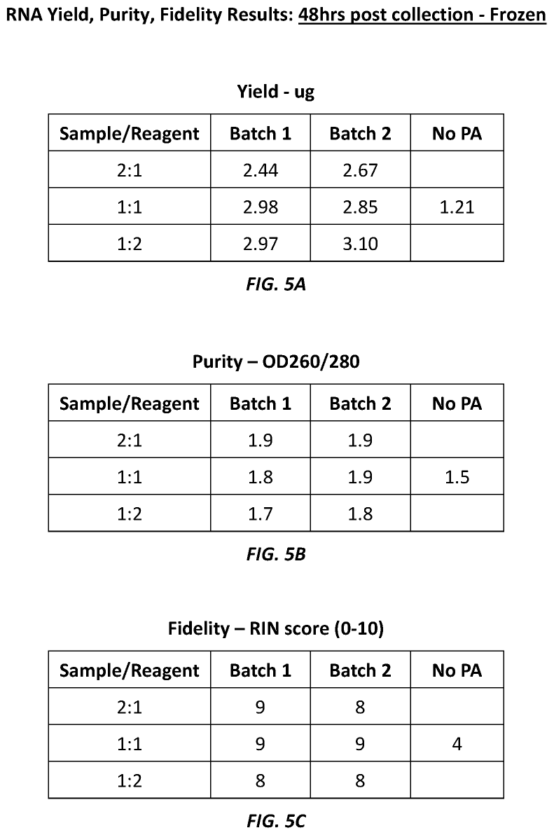

FIGS. 3A-3C, 4A-4C, and 5A-5C each illustrate Yield (A), Purity (B), and Fidelity (C) Results of RNA extracted from saliva samples immediately after collection (FIGS. 3A-3C), after being stored at room temperature for 48 hours (FIGS. 4A-4C), and after being stored frozen (−80° C.) for 48 hours (FIGS. 5A-5C) in RNA preservation composition 1.0. Experiments were completed with two independent batches of preservation agent (all of which were stored at room temperature and 4° C. for 30-60 days post creation). Experiments were performed using 5 independent subjects across all variables tested. Chemagen bead-based extraction chemistry optimized for whole blood DNA extraction was used for all RNA extractions. Several ratios of preservation agent to saliva were evaluated (2:1, 1:1, 1:2). Results indicate that storage in the inventive RNA preservation compositions may increase yield over immediate extraction, while maintaining purity and quality. Furthermore, a 1:1 ratio or a 1:2 ratio, v/v, of preservation agent to saliva, may be optimal in some embodiments.

### Kits

One or more embodiments of the present disclosure can comprise a kit, such as a biological sample collection and/or preservation kit. In one or more embodiments, the composition of the present disclosure can be included in or incorporated into the kit. Some embodiments can include a kit comprising a biological sample collection device (or container) and a composition of the present disclosure. Illustrative sample collection apparatus can include a container or vial (e.g., a tube) having a sample collection portion. For instance, the container can comprise an outer wall at least partially bounding an internal compartment. The container can also have an opening in fluidic communication with the compartment. The container can also have a cap for closing or sealing the opening of the apparatus.

In at least one embodiment, the composition can be disposed in a portion of a sample collection apparatus. Illustratively, the internal compartment can contain the composition, to which a biological sample can be added. Alternatively, the sample can be added to the compartment and the composition added to the sample post-collection. Accordingly, the collection device (or container) can be configured to receive the biological sample (e.g., in an inner compartment thereof) and have the composition added thereto. For instance, the apparatus can include a composition dispenser for adding the composition to the compartment, pre- or post-sample collection. For example, in some embodiments, the composition can be disposed in a portion of a cap or lid of the device. The cap can have a compartment for retaining the composition until it is to be added to the compartment of the container. In some embodiments, closing the lead can dispense the composition into the compartment, where the biological sample is disposed.

In some embodiments, the composition in the kit can be substantially free or devoid of microbial contamination (as described above).

### Methods of Manufacture

Some embodiments include a method of manufacturing a composition of the present disclosure. Embodiments can include providing or obtaining a carrier, as described herein. Embodiments can include adding to the carrier a suitable amount of one or more components or ingredients described herein (e.g., to a final concentration described herein). Embodiments can include adding to the carrier a described amount of stock solution of one or more components or ingredients described herein.

At least one embodiment includes adding to the carrier a chaotropic agent, buffering agent, chelating agent, surfactant, alcohol, and/or reducing agent. One or more embodiments can include adding to the carrier an optional acid and/or visual indicator.

At least one embodiment includes adding to a (liquid) carrier, a chaotropic agent to a final concentration of 1-10%, w/w, buffering agent to a final concentration of 1-10%, w/w, chelating agent to a final concentration of 0.01-2%, w/w, surfactant to a final concentration of 1-5%, w/w, alcohol to a final concentration of 5-30%, w/w, and/or reducing agent to a final concentration of 0.01-2%, w/w. At least one embodiment includes adding to a (liquid) carrier an optionally acid qs to pH 4-7 (or greater than 4 and below 7) and/or a visual indicator to a final concentration of 0.00005-0.5%, w/w. The carrier can be included at qs to 100%. Other concentrations, as described herein, are also contemplated.

In some embodiments, the chaotropic agent can be or comprise LiCl, the buffering agent can be or comprise sodium citrate, the chelating agent can be or comprise EDTA or EDTA disodium (salt) dihydrate, the surfactant can be or comprise SLS, the alcohol can be or comprise ethanol and/or isopropanol (e.g., SDA 3C), the reducing agent can be or comprise TCEP, the acid can be or comprise HCl, the carrier can be or comprise (RNAse-free) water, and/or the optional visual indicator can be or comprise FD&C Blue No. 1.

A method of manufacturing a nucleic acid or RNA stabilization and/or preservation composition can include adding the carrier to a vessel (e.g., charging a mixing tank with (filtered, deionized, etc.) water. In some embodiments, a mixer can be activated before one or more additional components or ingredients are added to the carrier. In some embodiments, a mixer can be activated after one or more additional components or ingredients are added to the carrier. In some embodiments, a mixer can be set to a speed setting of 2-8, preferably 3-7, more preferably 4-6, still more preferably 5 and/or sweep setting of 2-8, preferably 3-7, more preferably 4-6, still more preferably 5. In some embodiments, the carrier can be heated to a suitable mixing temperature before one or more additional components or ingredients are added to the carrier. In some embodiments, the carrier can be heated to a suitable mixing temperature after one or more additional components or ingredients are added to the carrier. In some embodiments, the suitable mixing temperature can be (about) 55-95±5° F., preferably 60-90±5° F., more preferably 65-85±5° F., still more preferably 70-80±5° F., most preferably 75±5° F.

In some embodiments, a suitable amount of chaotropic agent (e.g., LiCl) can be added to the carrier (e.g., to a final concentration of about 1%-10%, w/w of the composition). In some embodiments, the chaotropic agent can be mixed for a period of time (e.g., between 30-300 minutes, preferably 60-240 minutes, more preferably 120-180, still more preferably 140-160 minute, most preferably 150 minutes, or until the chaotropic agent is dissolved (in solution) in the carrier.

In some embodiments, a suitable amount of buffering agent (e.g., sodium citrate) can be added to the carrier (e.g., to a final concentration of about 1%-10%, w/w of the composition). In some embodiments, the buffering agent can be mixed in for a period of time (e.g., between 1-90 minutes, preferably 5-60 minutes, more preferably 10-45, still more preferably 12-30 minute, still more preferably 15-25 minute, most preferably (about) 20 minutes, or until the buffering agent is dissolved (in solution) in the carrier.

In some embodiments, a suitable amount of chelating agent (e.g., EDTA, EDTA disodium salt, EDTA disodium (salt) dihydrate) can be added to the carrier (e.g., to a final concentration of about 0.01%-2%, w/w of the composition). In some embodiments, the chelating agent can be mixed in for a period of time (e.g., between 1-90 minutes, preferably 5-60 minutes, more preferably 10-45, still more preferably 12-30 minute, still more preferably 15-25 minute, most preferably (about) 20 minutes, or until the chelating agent is dissolved (in solution) in the carrier. In at least one embodiment, the buffering agent and the chelating agent can be added to the carrier together, at (approximately) the same time, contemporarily, concomitantly, and/or (substantially) concurrently (or simultaneously), with or without being pre-mixed together. In some embodiments, the buffering agent and the chelating agent can be added to the carrier separately.

In some embodiments, a suitable amount of surfactant (e.g., SLS) can be added to the carrier (e.g., to a final concentration of about 1%-5%, w/w of the composition). In some embodiments, the surfactant can be mixed in for a period of time (e.g., between 1-90 minutes, preferably 5-60 minutes, more preferably 10-45, still more preferably 15-35 minute, still more preferably 20-30 minute, most preferably (about) 25 minutes, or until the surfactant is dissolved (in solution) in the carrier.

In some embodiments, a suitable amount of alcohol (e.g., ethanol, a mixture of ethanol and another chemical, such as isopropanol, or a SDA, preferably SDA 3C) can be added to the carrier (e.g., to a final concentration of about 5%-30%, w/w of the composition). In some embodiments, the alcohol can be mixed in for a period of time (e.g., between 5-90 minutes, preferably 10-75 minutes, more preferably 15-60, still more preferably 25-45 minute, still more preferably 30-40 minute, most preferably (about) 35 minutes, or until the alcohol is dissolved (in solution) in the carrier.

In some embodiments, a suitable amount of an optional visual indicator (e.g., a coloring agent, a dye, preferably a blue dye, such as FD&C Blue No. 1) can be added to the carrier (e.g., to a final concentration of about 0.00037%, w/w of the composition). In some embodiments, the visual indicator can be mixed in for a period of time (e.g., between 5-90 minutes, preferably 10-60 minutes, more preferably 15-45, still more preferably 10-30 minute, still more preferably 15-25 minute, most preferably (about) 20 minutes, or until the alcohol is dissolved (in solution) in the carrier.

In some embodiments, a suitable amount of an acid (e.g., hydrochloric acid) can be added to the carrier (e.g., qs to pH 4-7). In some embodiments, the acid can be mixed in for a period of time (e.g., between 5-90 minutes, preferably 10-60 minutes, more preferably 15-45, still more preferably 10-30 minute, still more preferably 15-25 minute, most preferably (about) 20 minutes, or until the acid is dissolved (in solution) in the carrier and/or the mixture equilibrates at the desired pH.

In some embodiments, a suitable amount of a mucolytic agent (or reducing agent) (e.g., TCEP) can be added to the carrier (e.g., to a final concentration of about 0.01%-2%, w/w of the composition). In some embodiments, the acid can be mixed in for a period of time (e.g., between 5-90 minutes, preferably 10-60 minutes, more preferably 15-45, still more preferably 10-30 minute, still more preferably 15-25 minute, most preferably (about) 20 minutes, or until the acid is dissolved (in solution) in the carrier and/or the mixture equilibrates at the desired pH.

An illustrative manufacturing method is provided below (referring to Tables 1-16).

Step 1. Combine ingredients 1-4 at ambient temperature in RNAse-free container.

Step 2. Mix ingredients for a minimum of 4 hours, or until visibly fully dissolved.

Step 3. Add ingredients 5-6 to the solution (item 5 not included in solution 1.0).

Step 4. Continue mixing for a minimum of 30 minutes, or until visibly fully dissolved.

Step 5. Check pH. Adjust to pH 5.5 with ingredient 7, and note amount used.

Step 6. Add ingredient 9 to the solution (ingredient 8 only used in solution 1.0).

Step 7. Continue mixing until completely dissolved.

Step 8. Adjust final volume to 150 mL with additional RNAse-free water (ingredient 1). Note amount used.

Step 9. Filter through 0.2 micron filter into a new, labeled, RNAse-free container (sterile filtration).

Step 10. Using a sealable lid, close the container. Solution may be stored at room temperature.

Quality control testing can be performed at any suitable point during manufacture. For example, upon completion of the bulk manufacturing process for each batch, two (2) samples (approximately 4 ounces each) were aseptically obtained from the bulk blend tank using clean and sanitized, approved and appropriate tools for obtaining samples from each of the following locations: top surface of batch near center of tank, top surface of batch near side wall of tank, middle of batch near center of tank, middle of batch near side wall of tank, bottom of batch near center of tank, and bottom of batch near side wall of tank. Each sample was placed in a sterile cup and labeled.

Each sample is tested for proper appearance, specific gravity, and pH. In addition, assays were performed to test concertation and/or effectiveness of the chelating agent, alcohol, and reducing agent. In addition, contamination (microbial limits) were tested by measuring total aerobic plate count, yeast and mold, Staphylococus aureus, and Pseudomonas aeruginosa. Table 17 presents testing specifications for various quality control measures.

TABLE 17

| TEST | METHOD | SPECIFICATION |
|---|---|---|
| Appearance | SOP 403 | Comparable to Standard |
| Specific gravity @ 25° C. | SOP 405 | Report only |
| pH | STM M403 | 7.9-8.3 |
| Assay - Disodium EDTA | Cornerstone | 0.73-0.89% |
| Assay - SDA Alcohol 3C | Cornerstone | 15.96-19.50% |
| Assay - N-Acetylcysteine | Cornerstone | 0.084-0.102% |
| Microbial limits | STM M429 | Less than 100 cfu/g |
| Yeast and mold | STM M429 | Less than 100 cfu/g |
| Staphylococcus aureus | STM M429 | Absence |
| Pseudomonas aeruginosa | STM M429 | Absence |

In some embodiments, the method can include sealing the composition in a suitable storage vessel or a portion of a sample collection apparatus (e.g., a composition storage portion of a container or vial (e.g., a tube). Samples were also subjected to controlled room temperature (CRT) and accelerated (ACC) stability testing in storage vessels and sample collection apparatus.

In some embodiments, the method can produce or result in a composition that can be substantially free or devoid of microbial contamination (as described above).

### Methods of Use

Some embodiments include a method of preserving and/or stabilizing ribonucleic acid. The method can comprise providing a biological sample containing the ribonucleic acid and combining a composition of the present disclosure with the biological sample. In at least one embodiment, the biological sample can be a mucin-containing bodily fluid or tissue, such as sputum or saliva. The method can include reducing the viscosity of a mucin-containing bodily fluid or tissue (e.g., by reducing disulfide bonds inherent to mucin with a mucolytic agent or reducing agent). In some embodiments, the biological sample can be a bodily fluid sample, such as a blood sample, cerebral spinal fluid sample, urine sample, and so forth. In some embodiments, the biological sample can be a cell sample, organ sample, or tissue sample. In some embodiments, the biological sample can be a cancer cell or tissue sample.

In at least one embodiment, the nucleic acid is RNA. In some embodiments, the composition can stabilize the nucleic acid or RNA (e.g., against degradation). Without being bound to any theory, RNA is known to be highly unstable and/or sensitive to degradation under certain conditions (e.g., in solution and/or when exposed to nuclease, unfavorable temperatures, UV light, and/or various chemicals). Stabilizing or preserving reagents (e.g., solutions) are needed to preserve and/or stabilize RNA in biological samples (such as a saliva sample) during storage, shipping, handling, and pre-processing steps to ensure survival of (at least a portion of) the RNA, until analysis can be performed thereof. RNA preservation or stabilization is generally considered to be much more difficult than DNA preservation or stabilization and existing RNA preservation and/or stabilization solutions may not be optimal for stabilizing RNA from certain biological samples, such as saliva, during transport, handling, and/or pre-processing, and/or for certain types of analytical techniques or devices for performing the same.

In some embodiments, the compositions of the present disclosure can stabilize

RNA (in solution and/or from a biological (e.g., saliva) sample) for a first period of time. In some embodiments, the first period of time can be greater than or equal to about 1 day, 2 days, 3 days 4 days, 5 days, 6 days, 7 days, 8 days, 9 days, 10 days, 11 days, 12 days, 13 days, 14 days, 21 days, 28 days, 30 days, 45 days, 60 days, 90 days, 120 days, 240 days, 300 days, or 365 days. In some embodiments, the composition can stabilize RNA (in solution and/or from a biological (e.g., saliva) sample) for the first period of time (i) at room temperature, between about 15° C. to 30° C., or between about 20° C. to 25° C., (ii) refrigerated, between about 1° C. to 20° C., or between about 1° C. to 15° C., (iii) frozen, between about −80° C. to 0° C., or between about -20° C. to 0° C., or (iv) other suitable temperature or temperature range. Indeed, compositions of the present disclosure have been shown to stabilize (a suitable amount of) RNA (in solution and/or from a biological (e.g., saliva) sample) for at least 7 days with refrigeration (e.g., between about 1° C. to 15° C., preferably between about 1° C. to 10° C., more preferably between about 1° C. to 8° C., still more preferably between about 1° C. to 6° C., still more preferably between about 2° C. to 6° C., most preferably about 4° C.). Moreover, compositions of the present disclosure have been shown to stabilize (a suitable amount of) RNA (in solution and/or from a biological (e.g., saliva) sample) for at least 5 days at room temperature (e.g., between about 15° C. to 30° C., preferably between about 20° C. to 25° C., more preferably between about 21° C. to 25° C., still more preferably between about 22° C. to 24° C., most preferably about 23° C.).

Notwithstanding the foregoing, suitable amounts of RNA appear to persist well beyond 7 days (refrigerated) and 5 days (at room temperature). While testing is ongoing, based on projection data (not shown), it is anticipated that the compositions of the present disclosure will be effective to stabilize (a suitable amount of) RNA (in solution and/or from a biological (e.g., saliva) sample) for at least 7 days, preferably at least 8 days, more preferably at least 9 days, still more preferably at least 10 days, still more preferably at least 11 days, still more preferably at least 12 days, still more preferably at least 13 days, still more preferably at least 14 days, at room temperature (e.g., between about 15° C. to 30° C., preferably between about 20° C. to 25° C., more preferably between about 21° C. to 25° C., still more preferably between about 22° C. to 24° C., most preferably about 23° C.), and for at least 10 days, preferably at least 11 days, more preferably at least 12 days, still more preferably at least 13 days, still more preferably at least 14 days, still more preferably at least 15 days, still more preferably at least 16 days, still more preferably at least 17 days, still more preferably at least 18 days, still more preferably at least 19 days, still more preferably at least 20 days, still more preferably at least 21 days with refrigeration (e.g., between about 1° C. to 15° C., preferably between about 1° C. to 10° C., more preferably between about 1° C. to 8° C., still more preferably between about 1° C. to 6° C., still more preferably between about 2° C. to 6° C., most preferably about 4° C.).

In some embodiments, compositions of the present disclosure can be stable (e.g., shelf stable) for a second period of time. In some embodiments, the second period of time can be greater than or equal to about 12 months, 18 months, 24 months, 30 months, or 36 months. In some embodiments, the composition can be stabile for the second period of time (i) at room temperature, between about 15° C. to 30° C., or between about 20° C. to 25° C., (ii) refrigerated, between about 1° C. to 20° C., or between about 1° C. to 15° C., (iii) frozen, between about −80° C. to 0° C., or between about −20° C. to 0° C., or (iv) other suitable temperature or temperature range. Indeed, compositions of the present disclosure have been shown to be (shelf) stable for at least 60 days with refrigeration (e.g., between about 1° C. to 15° C., preferably between about 1° C. to 10° C., more preferably between about 1° C. to 8° C., still more preferably between about 1° C. to 6° C., still more preferably between about 2° C. to 6° C., most preferably about 4° C.). Moreover, compositions of the present disclosure have been shown to be (shelf) stable for at least 30 days at room temperature (e.g., between about 15° C. to 30° C., preferably between about 20° C. to 25° C., more preferably between about 21° C. to 25° C., still more preferably between about 22° C. to 24° C., most preferably about 23° C.).

Notwithstanding the foregoing, compositions of the present disclosure appear to be (shelf) stable well beyond 60 days (refrigerated) and 30 days (at room temperature). It is noted that each of the components listed in the Example compositions of Tables 1-16 are inherently stable in water at the concentrations listed. In some embodiments, the concentration of each ingredient is intentionally maintained low enough to achieve long-term (shelf) stability at ambient or room temperature, as well as refrigerated. While testing is ongoing, it is anticipated that the compositions illustrated in Tables 1-16, as well as other compositions of the present disclosure, compositions similar thereto, and/or compositions comprising one or more ingredients (e.g., as disclosed as being combined in embodiments of the present disclosure) within the disclosed concentration range(s), will be (shelf) stable for at least a year at ambient or room temperature and refrigerated.

At least one embodiment includes a method of recovering a nucleic acid (e.g., RNA) from saliva or sputum, comprising: i) obtaining saliva or sputum from a subject, ii) contacting the saliva or sputum with a composition of the present disclosure to form a sample mixture, iii) optionally contacting the mixture with a protease, and iv) recovering nucleic acid (e.g., RNA) from the mixture. In some embodiments, the method can further comprise performing RNA analysis on the recovered RNA. In one or more embodiments, 1.5 mL of RNA preserving solution of the present disclosure (e.g., as illustratively presented in Tables 1-16) is added to 1.5 mL of (human) saliva or sputum.

At least one embodiment includes a method of recovering a nucleic acid (e.g., RNA) from a biological fluid sample, comprising: i) obtaining biological fluid from a subject, ii) contacting the biological fluid with a composition of the present disclosure to form a sample mixture, iii) optionally contacting the mixture with a protease, and iv) recovering the nucleic acid (e.g., RNA) from the mixture. In one or more embodiments, 1.5 mL of RNA preserving solution of the present disclosure (e.g., as illustratively presented in Tables 1-16) is added to 1.5 mL of (human) bodily fluid.

At least one embodiment includes a method of recovering a nucleic acid (e.g., RNA) from a tissue sample, comprising: i) obtaining tissue from a subject, ii) contacting the tissue with a composition of the present disclosure to form a sample mixture, iii) optionally contacting the mixture with a protease, and iv) recovering the nucleic acid (e.g., RNA) from the mixture. In one or more embodiments, 1.5 mL of RNA preserving solution of the present disclosure (e.g., as illustratively presented in Tables 1-16) a suitable volume or mass of tissue or cellular sample. In some embodiments, the tissue may be homogenized in presence of stabilizing solution.

In some embodiments, when added in a suitable amount to the biological sample, the compositions of the present disclosure do not significantly inhibit or interfere with subsequent nucleic acid analysis, such as, for example, direct detection of RNA (e.g., Nanostring), reverse transcription of RNA to cDNA, DNA amplification (e.g., via PCR), nucleic acid (DNA) sequencing (e.g., next generation (NextGen) sequencing, microarray analysis, and so forth, which can be useful for gene expression analysis.

### Sample Collection

Some embodiments of the present disclosure include obtaining, providing, and/or collecting a biological sample (e.g., from a subject, such as a human subject). In some embodiments, the biological sample can be or comprise (human) saliva, bodily fluid, tissue, etc. The (human) sample can be collected aseptically (to avoid (microbial) contamination). In one or more embodiments, the sample can be collected into a sample collection apparatus or sample container thereof. In some embodiments, the sample collection apparatus or container can be part of a kit and/or can include a composition of the present disclosure. Embodiments can include contacting the sample with a composition of the present disclosure.

### RNA Extraction and Analysis:

Some embodiments of the present disclosure include extracting nucleic acid (e.g., RNA) from the biological sample. The following is a non-exhaustive listing or description of various modes of extraction or extraction procedures that may be suitable for use with compositions of the present disclosure.

### Extraction Chemistry

Organic—Phenol chloroform extraction is a common procedure/mechanism employed in both research and clinical labs and is considered sample type dependent when it comes to tissue source. In some embodiments of the present disclosure, a (manual) phenol/chloroform extraction protocol, followed by a chloroform back extraction to help remove any organic solvent contamination, is and/or can be performed to extract total RNA.

Solid phase—without being bound to any theory, spin column and vacuum manifold solutions for binding DNA to a solid support for nucleic acid purification may be adapted for RNA purification. Once the RNA is attached to the support, a series of washes may be performed. Ultimately RNA may be eluted off of the solid support in a small volume for analysis. Spin column chemistry is frequently used in both the research and clinical lab.

Bead-based—Beads or (para)magnetic beads are prepared with various binding moieties or by charge in order to bind total RNA. The beads are captured by a magnetic field so anything unbound to the beads can be washed away as part of the purification process. Once washing is complete the nucleic acid is eluted off of the beads with a solution that solubilizes the RNA leaving the beads behind which are subsequently removed by reapplying a magnetic field. There are both small and large volume automated solutions for this approach in the research and clinical environment.

In some embodiments of the present disclosure, the binding buffer, or other buffers, reagents, etc., of one or more commercially available kit(s) or chemistries can be modified (e.g., optimized) to be compatible with one or more composition(s) of the present disclosure. Changes in pH and buffer composition, for example, can have a direct impact on the efficiency of RNA binding to the substrate which would affect extraction yields and, potentially, sample quality.

### Analytical Approaches

Some embodiments include processing and/or analyzing the extracted nucleic acids (e.g., RNA). Several methods are available for analyzing the extracted nucleic acids (e.g., RNA). The following is a non-exhaustive listing or description of various methods for analyzing the extracted nucleic acids (e.g., RNA) that may be suitable for use with compositions of the present disclosure.

### RT-PCR

Reverse Transcription Polymerase Chain Reaction (RT-PCR) analysis is a rapid and cost-effective means for qualitative and quantitative RNA analysis. RNA is reverse transcribed and amplified, optionally while monitoring amplification in real time (e.g., by dsDNA binding dye fluorescence). Alternatively, a series of PCR reactions (of varying size amplicons) are generated from all reverse transcribed cDNA templates and resolved via electrophoresis for the correct size amplification product. The range of PCR amplicon sizes will provide information on the fidelity of all RNA extraction products.

qPCR

Quantitative PCR (qPCR) uses dual labeled fluorogenic probes for the quantitation of PCR amplicons. Absolute and differential gene expression utilizing Taqman chemistry will be used to determine both the absolute and relative amount of RNA transcripts collected and extracted across all extraction approaches. Gene expression levels of the subjects will be measured for abundance and relative differences across all variables being analyzed. All quantitative measurements will be made in triplicate.

dPCR

Digital PCR (dPCR) is an emerging technology being employed for sensitive detection of gene expression targets in samples with limiting amounts and/or limiting quality. The same Taqman assays will be used to determine the absolute sensitivity of every RNA sample extracted. Given the sensitivity of dPCR we will be able to determine the ultimate detection level of each transcript being analyzed.

### Microarray

The measurement of RNA transcripts simultaneously has tremendous implications when it comes to both discovery and clinical classification of a single RNA sample. The sensitivity and specificity requirements are quite different than QPCR based analysis and the approach for gene expression quantitation is also different as this analytical approach uses a hybridization based mechanism for quantifying RNA transcripts. Relative gene expression levels across donors processed with different RNA extraction chemistries will be a critical analytical endpoint.

NextGen sequencing—RNA-Seq

As used herein, “next generation sequencing” (NGS), also known as high-throughput sequencing, refers to non-Sanger-based, high-throughput RNA sequencing technologies. Through NGS, millions or even billions of RNA strands can be sequenced in parallel, yielding substantially more throughput and minimizing the need for the fragment-cloning methods that are often used in Sanger sequencing of genomes. NGS is the catch-all term used to describe a number of different modern sequencing technologies or platforms including, for example, pyrosequencing, sequencing by synthesis, sequencing by ligation, ion semiconductor sequencing, and others as known in the art.

As understood by those skilled in the art, NGS generally allow sequencing of large amounts of RNA much more quickly and affordably than Sanger sequencing. In NGS, vast numbers of short reads are sequenced in a single stroke. To do this, firstly the input sample can be cleaved into short sections. The length of these sections depends on the particular sequencing machinery used. Illustrative examples of specific NGS technologies include, for example, Illumina® (SBS chemistry) sequencing, Ion torrent, S5 sequencing, and so forth.

NextGen technologies formerly offered by Roche and Life Technologies may also be suitable in certain embodiments. Without being bound to any theory, Roche 454 sequencing may be suitable for sequencing longer reads than Illumina®. Like Illumina®, it may do this by sequencing multiple reads at once by reading optical signals as bases are added. As in Illumina®, the DNA or RNA is fragmented into shorter reads, in this case up to 1 kb. Generic adaptors are added to the ends and these are annealed to beads, one DNA fragment per bead. The fragments are then amplified by PCR using adaptor-specific primers. Each bead is then placed in a single well of a slide. So each well will contain a single bead, covered in many PCR copies of a single sequence. The wells also contain DNA polymerase and sequencing buffers. The slide is flooded with one of the four NTP species. Where this nucleotide is next in the sequence, it is added to the sequence read. If that single base repeats, then more will be added. So if we flood with Guanine bases, and the next in a sequence is G, one G will be added, however if the next part of the sequence is GGGG, then four Gs will be added. The addition of each nucleotide releases a light signal. These locations of signals are detected and used to determine which beads the nucleotides are added to. This NTP mix is washed away. The next NTP mix is now added and the process repeated, cycling through the four NTPs. This kind of sequencing generates graphs for each sequence read, showing the signal density for each nucleotide wash. The sequence can then be determined computationally from the signal density in each wash. All of the sequence reads we get from 454 will be different lengths, because different numbers of bases will be added with each cycle.

In Illumina sequencing, 100-150 bp reads are used. Somewhat longer fragments are ligated to generic adaptors and annealed to a slide using the adaptors. PCR is carried out to amplify each read, creating a spot with many copies of the same read. They are then separated into single strands to be sequenced. The slide is flooded with nucleotides and DNA polymerase. These nucleotides are fluorescently labelled, with the color corresponding to the base. They also have a terminator, so that only one base is added at a time. An image is taken of the slide. In each read location, there will be a fluorescent signal indicating the base that has been added. The slide is then prepared for the next cycle. The terminators are removed, allowing the next base to be added, and the fluorescent signal is removed, preventing the signal from contaminating the next image. The process is repeated, adding one nucleotide at a time and imaging in between. Computers are then used to detect the base at each site in each image and these are used to construct a sequence. All of the sequence reads will be the same length, as the read length depends on the number of cycles carried out.

Unlike Illumina® (and Roche 454), Ion torrent and S5 sequencing do not make use of optical signals. Instead, they exploit the fact that addition of a dNTP to a DNA polymer releases an H+ ion. As in other kinds of NGS, the input DNA or RNA is fragmented, this time ˜200 bp. Adaptors are added and one molecule is placed onto a bead. The molecules are amplified on the bead by emulsion PCR. Each bead is placed into a single well of a slide. Like Roche 454, the slide is flooded with a single species of dNTP, along with buffers and polymerase, one NTP at a time. The pH is detected is each of the wells, as each H+ ion released will decrease the pH. The changes in pH allow us to determine if that base, and how many thereof, was added to the sequence read. The dNTPs are washed away, and the process is repeated cycling through the different dNTP species. The pH change, if any, is used to determine how many bases (if any) were added with each cycle.

Additionally, or alternatively, the sequencing may be more generally performed by a fluorescent-based sequencing technique and/or any electrical-current-based sequencing technique. Illustrative examples of fluorescent-based sequencing techniques include any technique that incorporates nucleotides conjugated to a fluorophore, such as, for example sequencing using Illumina® based sequencing methods and systems. Illustrative examples of electrical-current-based sequencing techniques include any sequencing technique (including strand sequencing methods) that measures the electrical current of a polynucleotide as it passes through a pore inserted into a charged membrane or otherwise specifically disrupts the electrical current of a sensor and/or charged membrane.

In some embodiments, direct detection NGS (e.g., Oxford Nanopore, PacBio), utilizing long read technology, for example, may also be suitable in certain embodiments. A non-limiting example of direct detection NGS techniques include the Nanopore DNA sequencing systems and methods of Oxford NanoPore Technologies®.

As an illustrative example, a sequencing run that generates data having 0.5× coverage will theoretically leave half of the sample unrepresented. Using sequencing methods that “chop up” the nucleic acid into small fragments for sequencing, the final product may be a sequence library representing about half of the total reference genome, where an aligned reference genome is littered with a smattering of smaller nucleic acid matches. On the other hand, using a strand sequencing method, again at low coverage (e.g., 0.5×), the result may be a sequence library representing, again, about half of the total reference genome. However, when aligned with a reference genome, the matching portions are much longer and may provide more definitive information, such as what sequences have been deleted, duplicated, inserted, etc. This may also prove problematic. While a longer contiguous portion of the genome may be represented by a strand sequencing approach, long contiguous portions of the genome are also left unknown. So, although strand sequencing methods may allow for a higher definition view of portions of the genome, smaller sequencing reads have the potential to provide a more global picture of the entire genome. In in this and other ways, strand sequencing may provide a robust model for analyzing copy number variation.

Though the foregoing is illustrative of known sequencing techniques and their applications to the inventive methods and systems disclosed herein, it should be understood that this does not preclude as yet undiscovered or otherwise undisclosed sequencing methods from being applied within the scope of the present invention. That is, the sequencing method, itself, is not, in many embodiments, a requisite inventive step (unless, for example, an improvement is provided to the method and/or system through use of a particular sequencing technique); rather, what is done with the sequencing data provided by the sequencing method and/or how those data are applied generally comprises an inventive step. Accordingly, it should be appreciated that future sequencing technologies (and those sequencing technologies that have not been explicitly listed herein), if used as a tool in the disclosed method or systems, are included within the scope of this application.

Additionally, any of the foregoing sequencing techniques may be used in any number or capacity and with any number of flow cells or other similar inputs that affect the total number of sequencing reads provided for each sequencing reaction/run.

Next Generation sequencing may ultimately become the standard for analysis of both DNA and RNA targets. A targeted panel including RNA transcripts covered by qPCR, dPCR and array based targets is created for all RNA samples through a standard library preparation process. Samples are barcoded and multiplexed on a NextGen platform for variant analysis. Data is de-multiplexed and analyzed for direct comparison of genotype call across all other platforms.

### Conclusion

Compositions of the present disclosure are surprisingly, significantly superior to existing RNA preservation products. In particular, it was surprising and unexpected that the compositions of the present disclosure work so well (e.g., yield high amounts of (human) ribonucleic acid (RNA) and/or have or exhibit low levels of microbial contamination). It was further surprising and unexpected that the compositions of the present disclosure work so well with the low amount of alcohol provided in some embodiments. For instance, in some embodiments, the amount of alcohol included in the composition can be less (e.g., about 20%, 25%, 30%, 35%, 40%, 45%, 50%, 55%, or 60% less) than typical, traditional, or existing nucleic acid or RNA preservation solutions. In addition, the lower amount of alcohol of more economical and/or makes the composition more amendable to shipping or transport (e.g., by more easily complying with shipping requirements and regulations, reducing volatility, etc.).

It was further surprising and unexpected that certain components or ingredients were as suitable, if not more suitable, for use in an RNA preservation solution, than more popular components or ingredients. For example, in some embodiments, Lithium Chloride (LiCl) was found to be a more effective chaotropic agent than the more common guanidine thiocyanate, guanidine isocyanate, and guanidine hydrochloride, for preservation of human RNA from a saliva sample. Similarly, in some embodiments, cetyltrimethylammonium bromide (CTAB) was found to be as effective of a detergent as the (stronger) detergents SDS, SLS, etc. in the preservation of human RNA from a saliva sample. It was further surprising and unexpected that a pH of about 5.5 was optimal in some embodiments of the RNA preservation solution. It was further surprising and unexpected that sodium citrate (trisodium citrate dihydrate) was as suitable, if not more suitable, for use in an RNA preservation solution, than more popular buffers, such as Tris, Tris-HC, Trizma® base, citrate, MES, BES, Bis-Tris, HEPES, MOPS, Bicine, Tricine, ADA, ACES, PIPES, bicarbonate, phosphate, TAE, TBE, sodium borate, sodium cacodylate. Inclusion of a specially denatured alcohol (e.g., SDA 3C) also carries the benefit of avoid various regulatory compliance issues and associate costs, while maintaining the effectiveness of ethanol.

It will be appreciated that certain embodiments (e.g., compositions, kits, method, etc.) may include, incorporate, or otherwise comprise features (e.g., properties, components, ingredients, elements, parts, portions, steps, etc.) described in other embodiments disclosed and/or described herein. Accordingly, the various features of one embodiment can be compatible with, combined with, included in, and/or incorporated into other embodiments of the present disclosure. Disclosure of certain features relative to one embodiment of the present disclosure should not be construed as limiting application or inclusion of said features to the specific embodiment. Rather, it will be appreciated that other embodiments can also include said features without necessarily departing from the scope of the present disclosure. Moreover, unless a feature is described as requiring another features in combination therewith, any feature described herein may be combined with any other feature of a same or different embodiment disclosed herein.

The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. Various alterations and/or modifications and additional applications of the features illustrated herein which would occur to one skilled in the relevant art and having possession of this disclosure, can be made to the illustrated embodiments without departing from the spirit and scope of the invention as defined by the claims, and are to be considered within the scope of this disclosure. While various features and embodiments have been disclosed herein, other features and embodiments are contemplated. For instance, well-known features and embodiments are not described herein in particular detail in order to avoid obscuring aspects of the described embodiments. Such features and embodiments are, however, also contemplated herein.

1. A ribonucleic acid (RNA) preservation composition, consisting essentially of:
about 3.33-6.66%, w/w, lithium chloride (LiC1);
about 1-2%, w/w, trisodium citrate dihydrate;
about 0.2-0.5%, w/w, ethylenediaminetetraacetic acid (EDTA) disodium dihydrate;
about 3-4%, w/w, N-lauroylsarcosine sodium salt (SLS) or cetyltrimethylammonium bromide (CTAB);
about 5-20%, w/w, alcohol, consisting essentially of about 95%, v/v, ethanol and about 5%, v/v, isopropanol;
about 0.15-0.3%, w/w, tris(2-carboxyethyl)phosphine hydrochloride (TCEP);
optionally, a colored dye; and
RNAse-free water q.s. to 100%,
the composition having a pH of about 5.5.

2.-20. (canceled)
